(19) United States
(12) Patent Application Publication
(10) Pub. No.: US 2024/0352020 A1
(43) Pub. Date: Oct. 24, 2024

(54) PYRAZOLE-CONTAINING CBP/CATENIN ANTAGONISTS AND USES THEREOF

(71) Applicant: 3+2 PHARMA, LLC, Lewes, DE (US)

(72) Inventor: Fuqiang Ruan, Bellevue, WA (US)

(21) Appl. No.: 18/684,619

(22) PCT Filed: Aug. 8, 2022
(86) PCT No.: PCT/US2022/039730

§ 371 Date: Feb. 16, 2024

Related U.S. Application Data
(60) Provisional application No. 63/237,246, filed on Aug. 26, 2021.

(51) Int. Cl. C07D 487/04; A61K 8/49; A61K 31/53; A61K 31/5377; A61K 45/06; A61Q 7/00; A61Q 19/08

(52) U.S. Cl. CPC C07D 487/04; A61K 8/4966; A61K 31/53; A61K 31/5377; A61K 45/06; A61Q 7/00; A61Q 19/08

(57) ABSTRACT

Provided are compounds of formula (la) and (lb), and pharmaceutically acceptable salts thereof. Additionally provided are compositions and pharmaceutical compositions comprising the compounds, therapeutic methods using same for modulating (e.g., inhibiting) CREB binding protein (CBP)/β-catenin mediated signaling in treating a condition, disease or disorder (e.g., fibrosis, cancer, neurological conditions, metabolic disorders (e.g., diabetes, etc.), and skin conditions (dermatitis, psoriasis, scarring, alopecia, etc.) mediated by aberrant CBP/β-catenin signaling, and cosmetic methods for treating skin conditions (e.g., aging, etc.). Additionally provided are methods for enhancing vaccine efficacy using the compounds and compositions. Further provided are methods for efficiently synthesizing an antagonist of CBP/catenin signaling pathway, comprising use, in a penultimate, or last reaction step, of an intermediate 2-propynyl-compound to form a pyrazole derivative (e.g., via 3+2 cycloaddition).

### CROSS-REFERENCE TO RELATED APPLICATION

This application claims the benefit of priority to U.S. Provisional Patent Application Ser. No. 63/237,246, filed Aug. 26, 2021, which is hereby incorporated by reference in its entirety.

### FIELD

Aspects of the invention relate generally to modulation of the Wnt/β-catenin pathway in mammalian (both human and non-human) cells and tissues, more particularly to small molecule inhibitors of CREB binding protein (CBP)/β-catenin signaling, and these small molecule CBP/β-catenin inhibitors having broad utility for modulating and treating CBP/β-catenin signaling-mediated conditions and disorders, including but not limited to one or more of fibrosis, cancer, neurological disorders, metabolic disorders (including diabetes and fatty liver disease, e.g., alcoholic (ALD) and non-alcoholic hepatic steatosis (ALD and NAFLD, respectively), and including non-alcoholic steatohepatitis (NASH)), skin conditions (e.g., dermatitis, psoriasis, alopecia, aging etc.), wound healing, aging, and optionally further including one or more of pulmonary hypertension, congestive heart failure, chronic kidney disease, renal fibrosis, endometriosis, cardiac fibrosis, polycystic ovary syndrome (PCOS), and/or systemic fibrosis/scleroderma. Additional aspects relate to enhancing vaccine efficacy using the disclosed compounds and compositions.

### BACKGROUND

The evolutionarily conserved Wnt/β-catenin signaling pathway plays fundamental and essential roles in both embryonic development and adult homeostasis. Additionally, given the established and critical roles of dysregulated/hyperactive CBP/β-catenin signaling in fibrosis, cancer, neurological disorders, skin disorders, and metabolic disorders (including diabetes and fatty liver disease) and aging, and in other Wnt/β-catenin-mediated conditions and disorders, there has been considerable interest in pursuing both therapeutic and cosmetic intervention by modulating (e.g., inhibiting) the CBP/β-catenin signaling, and/or increasing p300/β-catenin signaling, preferably using small molecule inhibitors of the CBP/β-catenin interaction. Developing specific small molecule inhibitors of the CBP/β-catenin interaction that are both sufficiently active and bioavailable (preferably orally available in many instances for therapeutic applications), however, remains a challenge that has, until now, substantially impeded realization of their therapeutic and cosmeceutical potential.

### SUMMARY

Aspects of the present invention are directed to small molecule inhibitors of the CBP/β-catenin interaction, along with compositions, pharmaceutical compositions comprising the compounds, and methods for synthesizing and using the compounds and compositions, both therapeutically and cosmeceutically, as further described below.

Aspects of the invention may include a compound of formula (Ia):

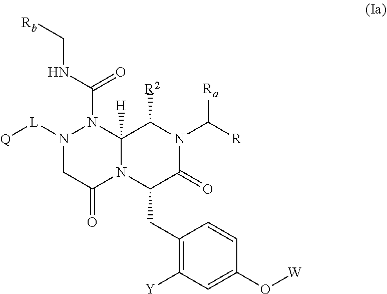

and pharmaceutically acceptable salts thereof, wherein:
Rₐ is hydrogen or —CH₃;
R_{b} is a monocyclic aryl group having 5 to 7 ring members, which may have 1 to 2 heteroatoms selected from nitrogen, oxygen or sulfur, and which may have one or more substituents selected from a group consisting of halide, cyano, lower alkyl, and —OC₁-C₈ alkyl;
R is a phenyl group; a substituted phenyl group having one or more substituents wherein the one or more substituents are independently selected from one or more of deuterium, amino, amidino, guanidino, hydrazino, amidazonyl, C₁₋₄alkylamino, C₁₋₄ dialkylamino, halogen, perfluoro C₁₋₄alkyl, C₁₋₄alkyl, C₁₋₃alkoxy, nitro, carboxy, cyano, sulfuryl, and hydroxyl groups; a benzyl group; a substituted benzyl group with one or more substituents where the one or more substituents are independently selected from one or more of deuterium, amino, amidino, guanidino, hydrazino, amidazonyl, C₁₋₄ alkylamino, C₁₋₄dialkylamino, halogen, perfluoro C₁₋₄alkyl, C₁₋₃alkoxy, nitro, carboxy, cyano, sulfuryl, and hydroxyl group; a monocyclic aryl group having 5 to 7 ring members, which may have 1 to 2 heteroatoms selected from nitrogen, oxygen or sulfur, having one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; or a bicyclic aryl group, or substituted bicyclic aryl, having 8 to 11 ring members, which may have 1 to 3 heteroatoms selected from nitrogen, oxygen or sulfur, where the substituted bicyclic aryl ring may have one or more substituents independently selected from one or more of deuterium, amino, amidino, guanidino, hydrazino, amidazonyl, C₁₋₄alkylamino, C₁₋₄ dialkylamino, halogen, perfluoro C₁₋₄alkyl, C₁₋₃alkoxy, nitro, carboxy, cyano, sulfuryl, and hydroxyl group;
R² is hydrogen, or —CH₃;
Y is selected from: hydrogen, deuterium, or halogen;
W is hydrogen, phosphate or phosphate salt, an ester of an alkyl acid or of a fatty acid;
L is —CH₂—, —CF₂—, or —C(CH₃)₂—; and
Q is a 5-membered nitrogen-containing heterocycle selected from:

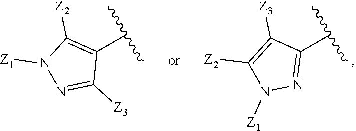

wherein Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C_{B} alkyl, —OC₁-C₆ alkyl-C(O)NH—OH, —NH₂, —C(O)NH—C₁-C₆ alkyl-heteroaryl, —NHC(O)C₁-C₆ alkyl-C(O)NH—OH, heteroaryl, cycloalkyl, heterocycloalkyl. Z₂ is independently selected from hydrogen, deuterium, halogen, C₁-C₄ alkyl, aryl, C₁-C₄ alkyl-C(O)—, aryl-C(O)—, C₁-C₄ alkyl-S—, aryl-S—, —C(O)O—C₁-C₄ alkyl, —CF₃. Z₃ is independently selected from hydrogen, deuterium, C₁-C₄ alkyl, —C(O)O—C₁-C₄ alkyl, —C(O)NH—C₁-C₄ alkyl.

In some aspects, W is selected from:

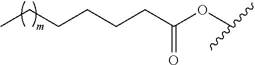

wherein m is 1 to 14,

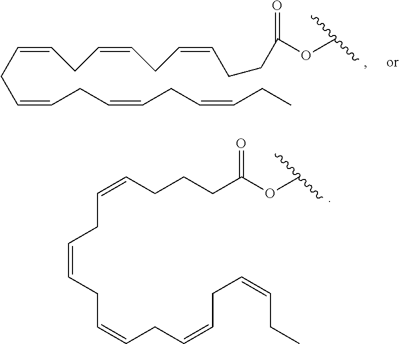

In some aspects, R is a bicyclic aryl selected from naphthyl, quinolinyl, isoquinolinyl, quinoxaline, phthalazine, quinazoline, cinnoline, or naphthyridine, or substituted variants thereof.

In some aspects, the compound is of the formula (Ib):

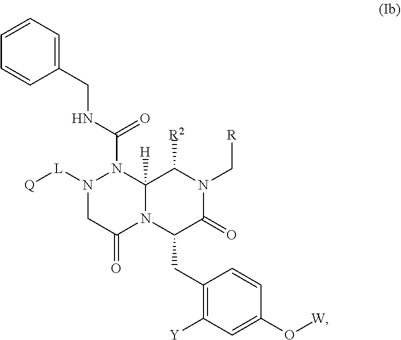

wherein R is as defined above.

In some aspects, L is —CH₂—;
Q is

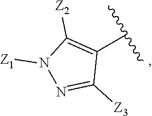

wherein Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C₆ alkyl, —OC₁-C₆ alkyl-C(O)NH—OH, —NH₂, —C(O)NH—C₁-C₈ alkyl-heteroaryl, —NHC(O)C₁-C₆ alkyl-C(O)NH—OH, heteroaryl, cycloalkyl, heterocycloalkyl. Z₂ and Z₃ are hydrogen, or deuterium. W is hydrogen, phosphate or phosphate salt, an ester of an alkyl acid or of a fatty acid.

In some aspects, the compound is:

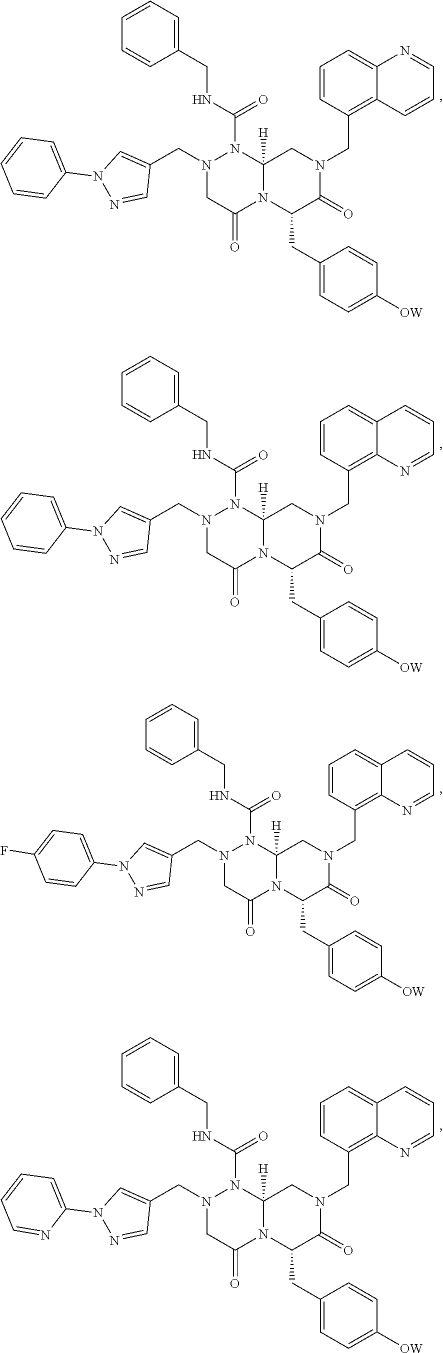

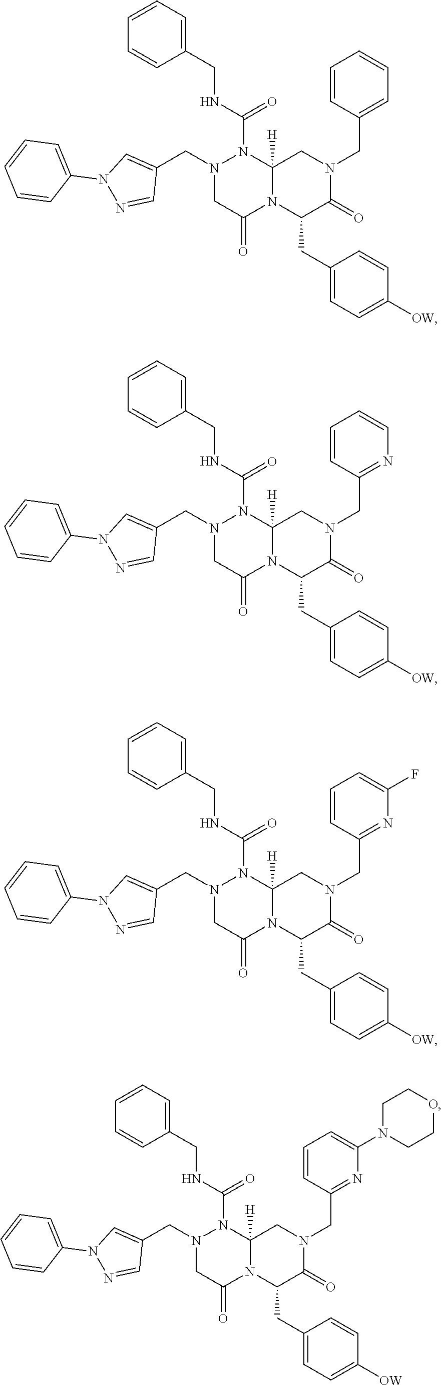

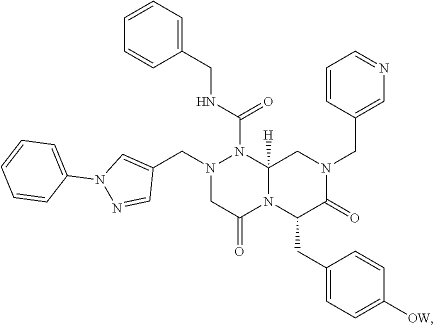

wherein W is as defined above.

In some aspects, the invention is directed to a composition, or pharmaceutical composition comprising any one of the previously discussed compounds, and a pharmaceutically acceptable carrier.

In some aspects, the invention is directed to method of treating a disease or disorder, comprising administering to a patient or a warm-blooded mammal, having a disease or disorder mediated by aberrant CREB binding protein (CBP)/β-catenin signaling, an amount of any one of the previously discussed compounds sufficient to inhibit the CBP/catenin mediated signaling, and/or enhance p300/catenin mediated signaling.

In some aspects, an amount of the administered compound comprises a therapeutically effective amount.

In some aspects, the disease or disorder comprises one or more of fibrosis, cancer, neurological conditions, metabolic disorders, and skin conditions.

In some aspects, the metabolic disorder comprises one or more of diabetes and/or fatty liver disease.

In some aspects, the fatty liver disease comprises one or more of alcoholic hepatic steatosis (ALD), non-alcoholic hepatic steatosis (NAFLD), and/or non-alcoholic steatohepatitis (NASH).

In some aspects, the fibrosis is fibrosis of the lung, liver, kidney, heart, endometrium, skin or systemic fibrosis.

In some aspects, the fibrosis comprises fibrosis in a SARS-CoV-2 (COVID-19) patient tissue.

In some aspects, treating cancer comprises administering the CBP/β-catenin antagonist in combination with, or as an adjunctive therapy with, one or more of cytotoxic and/or directed chemotherapy, and/or radiotherapy, and/or immunotherapy, including checkpoint inhibition (e.g., with anti-PD1, anti-PD-L1 or anti-CTLA4, etc.), chimeric antigen receptor (CAR-T) and/or CAR-NK cell based therapy.

In some aspects, the neurological condition comprises one or more of Huntington's (HD), Parkinson's (PD), Alzheimer's (AD), Multiple sclerosis (MS), and/or amyotrophic lateral sclerosis (ALS), muscular dystrophy (MD), and/or spinal muscular atrophy (SMA).

In some aspects, the skin condition comprises one or more of atopic dermatitis, psoriasis, acne, fibrosis, wounding, scarring, burns, sun or U.V. damage, diabetic ulceration, chronic ulceration, and/or alopecia.

In some aspects, W is an ester of an alkyl acid or of a fatty acid, and wherein administration comprises topical administration.

In some aspects, the invention is directed to a cosmetic method for treating a skin condition, comprising administering to a patient or a warm-blooded mammal, having a skin condition, a cosmeceutically effective amount of any one of the previously discussed compounds, wherein W is an ester of an alkyl acid or of a fatty acid, and wherein administration comprises topical administration.

In some aspects, the skin condition comprises one or more aging skin conditions selected from wrinkles, hyperpigmentation, redness, rosacea, dryness, cracking, loss of vibrance, loss of elasticity, thinning, loss of vibrance, scarring, acne, sun damage, hair loss, loss of hair coloration, reduced cuticle growth, reduced nail growth.

In some aspects, the invention is directed to a method for efficiently synthesizing a clinical grade drug, comprising use, in a penultimate or last reaction step under GMP conditions, of an intermediate 2-propynyl-compound to form a clinical grade pyrazole derivative via 3+2 cycloaddition.

In some aspects, the method includes enhancing vaccine efficacy, comprising, administering to a subject, prior to, and/or during, and/or after vaccination, an amount of any one of the previously disclosed compounds sufficient to inhibit CBP/β-catenin mediated signaling and/or enhance p300/catenin mediated signaling.

In some aspects, the amount of the administered compound comprises a therapeutically effective amount.

In some aspects, enhancing vaccine efficacy comprises one or more of: increased levels of vaccine antigen-specific antibodies; an increase in the percent protection afforded; an increase in the number or and/or persistence of differentiated memory T-cells; and/or an increase in the duration of protection.

In some aspects, inhibiting the CBP/β-catenin mediated signaling and/or enhancing the p300/catenin mediated signaling comprises one or more of: metabolic maintenance of cell asymmetry following division in activated T cells in the subject; enhancing antigen-specific immunity by increasing the number and/or persistence of differentiated memory T-cells; and/or enhancing the presentation of antigens to T-cells by antigen presenting cells to enhance cooperativity between the innate and acquired immune systems.

In some aspects, the vaccination, comprises administration of an anti-viral vaccine.

In some aspects, the vaccination comprises administration of an anti-viral vaccine selected from influenza, SARS, SARS-CoV-2, HPV-A, HPV-B, and/or Herpes Zoster.

In some aspects, the subject is a human having an age of 55-75 yr, 55-85 yr, ≥50 yr, ≥60 yr, or ≥65 yrs).

In some aspects, administration comprises: administration as a primer before vaccination; and/or co-administration with vaccination; and/or administration or co-administration subsequent to initial vaccine.

The above summary does not include an exhaustive list of all aspects of the present invention. It is contemplated that the invention includes all compositions and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

The aspects are illustrated by way of example and not by way of limitation in the figures of the accompanying drawings in which like references indicate similar elements. It should be noted that references to “an” or “one” aspect in this disclosure are not necessarily to the same aspect, and they mean at least one.

FIG. 1 shows, according to non-limiting aspects of the present invention, results of a SuperTOPFLASH cell-based luciferase assay (Wnt-driven Luciferase Activity in Stably Transfected Cell Line, Hek293, STF1.1), comparing the CBP/β-Catenin inhibition activities of two exemplary 1, 4-pyrazole containing compounds, 1 (wherein Z₂ and Z₃ are H) and 2 (wherein Z₂ is Br, and Z₃ is H) of the present invention; each compared in concentrations between 0.078 μM and 10 μM, with the art-recognized specific CBP/β-Catenin inhibitor ICG-001 used as a positive control (at 0.62, 1.25, 2.5, 5, 10 and 20 μM). As is readily apparent, the two inventive pyrazole-containing compounds generally have greater potency than that of ICG-001, and compound 1 has greater potency than that of compound 2 in this assay.

FIG. 2 shows, according to non-limiting aspects of the present invention, results of a SuperTOPFLASH cell-based luciferase assay (Wnt-driven Luciferase Activity in Stably Transfected Cell Line, Hek293, STF1.1), comparing the CBP/β-Catenin inhibition activities of two exemplary pyrazole containing compounds 3 (1, 3-pyrazole, wherein Z₂ and Z₃ are H) and 4 (1, 4-pyrazole, wherein Z₂ and Z₃ are H) of the present invention; each compared in concentrations between 0.625 μM and 5 μM, with the art-recognized specific CBP/β-Catenin inhibitor ICG-001 used as a positive control (at 2.5, 5 and 10 μM). As is readily apparent, the two inventive pyrazole-containing compounds generally have greater potency than that of ICG-001, and compound 4 has greater potency than that of compound 3 in this assay.

### DETAILED DESCRIPTION

In this section we shall explain several preferred aspects of this invention with reference to the appended drawings. Whenever aspects are not clearly defined, the scope of the invention is not limited only to that shown in the drawings, which are meant merely for the purpose of illustration. Also, while numerous details are set forth, it is understood that some aspects of the invention may be practiced without these details. In other instances, well-known compositions, components and/or techniques have not been shown in detail so as not to obscure the understanding of this description.

As used herein, the singular forms “a”, “an”, and “the” are intended to include the plural forms as well, unless the context indicates otherwise. It will be further understood that the terms “comprises” and/or “comprising” specify the presence of stated features, steps, operations, elements, compositions and/or components, but do not preclude the presence or addition of one or more other features, steps, operations, elements, components, compositions and/or groups thereof.

The terms “or” and “and/or” as used herein are to be interpreted as inclusive or meaning any one or any combination.

Incorporation by reference Unless stated otherwise, all references cited herein are incorporated by reference in their entirety.

Provided are compounds of formula (Ia):

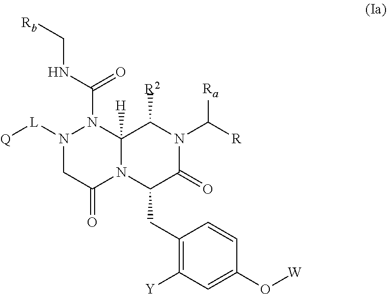

and stereoisomers, geometric isomers, tautomers, solvates (e.g., hydrates) metabolites, prodrugs, isotopically-labeled derivatives, and any salts including pharmaceutically acceptable salts thereof, wherein:
Rₐ is hydrogen or —CH₃;
R_{b} is a monocyclic aryl group having 5 to 7 ring members, which may have 1 to 2 heteroatoms selected from nitrogen, oxygen or sulfur, and which may have one or more substituents selected from a group consisting of halide, cyano, lower alkyl, and —OC₁-C₆ alkyl;
R is a phenyl group; a substituted phenyl group having one or more substituents wherein the one or more substituents are independently selected from one or more of deuterium, amino, amidino, guanidino, hydrazino, amidazonyl, C₁₋₄alkylamino, C₁₋₄ dialkylamino, halogen, perfluoro C₁₋₄alkyl, C₁₋₄alkyl, C₁₋₃alkoxy, nitro, carboxy, cyano, sulfuryl, and hydroxyl groups; a benzyl group; a substituted benzyl group with one or more substituents where the one or more substituents are independently selected from one or more of deuterium, amino, amidino, guanidino, hydrazino, amidazonyl, C₁₋₄ alkylamino, C₁₋₄dialkylamino, halogen, perfluoro C₁₋₄alkyl, C₁₋₃alkoxy, nitro, carboxy, cyano, sulfuryl, and hydroxyl group; a monocyclic aryl group having 5 to 7 ring members, which may have 1 to 2 heteroatoms selected from nitrogen, oxygen or sulfur, having one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; or a bicyclic aryl group, or substituted bicyclic aryl, having 8 to 11 ring members, which may have 1 to 3 heteroatoms selected from nitrogen, oxygen or sulfur, where the substituted bicyclic aryl ring may have one or more substituents independently selected from one or more of deuterium, amino, amidino, guanidino, hydrazino, amidazonyl, C₁₋₄alkylamino, C₁₋₄ dialkylamino, halogen, perfluoro C₁₋₄alkyl, C₁₋₃alkoxy, nitro, carboxy, cyano, sulfuryl, and hydroxyl group;
R² is hydrogen, or —CH₃;
Y is selected from: hydrogen, deuterium, or halogen;
W is hydrogen, phosphate or phosphate salt, an ester of an alkyl acid or of a fatty acid;
L is —CH₂—, —CF₂—, or —C(CH₃)₂—; and
Q is a 5-membered nitrogen-containing heteroaryl selected from:

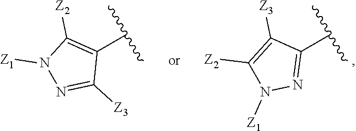

wherein Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C₆ alkyl, —OC₁-C₆ alkyl-C(O)NH—OH, —NH₂, —C(O)NH—C₁-C₆ alkyl-heteroaryl, —NHC(O)C₁-C₆ alkyl-C(O)NH—OH, heteroaryl, cycloalkyl, heterocycloalkyl. Z₂ is independently selected from hydrogen, deuterium, halogen, C₁-C₄ alkyl, aryl, C₁-C₄ alkyl-C(O)—, aryl-C(O)—, C₁-C₄ alkyl-S—, aryl-S—, —C(O)O—C₁-C₄ alkyl, —CF₃. Z₃ is independently selected from hydrogen, deuterium, C₁-C₄ alkyl, —C(O)O—C₁-C₄ alkyl, —C(O)NH—C₁-C₄ alkyl.

In the compounds, the ester of the alkyl acid or of the fatty acid may be preferably selected from:

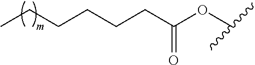

wherein m is 1 to 14,

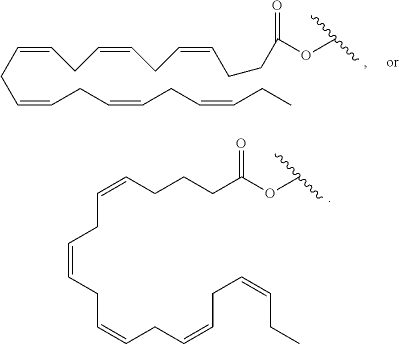

In the compounds, R may be a bicyclic aryl selected from naphthyl, quinolinyl, isoquinolinyl, quinoxaline, phthalazine, quinazoline, cinnoline, naphthyridine, or substituted variants thereof.

In the compounds, the compound may preferably be of the formula (Ib):

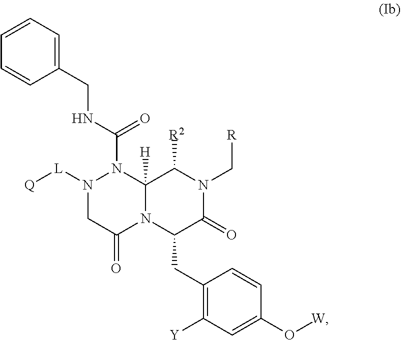

wherein R is as defined above.

In preferred aspects of the compounds:
L is —CH₂—;
Q is

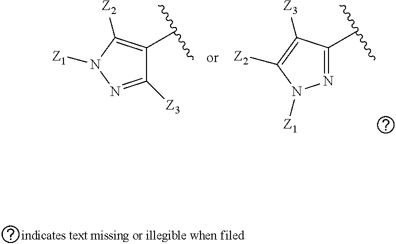

In the compounds, preferably, Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C₆ alkyl, —OC₁-C₆ alkyl-C(O)NH—OH, —NH₂, —C(O)NH—C₁-C₆ alkyl-heteroaryl, —NHC(O)C₁-C₆ alkyl-C(O)NH—OH, heteroaryl, cycloalkyl, heterocycloalkyl. Z₂ and Z₃ are independently selected from hydrogen, deuterium.

Another aspect of the present invention provides compounds of formula (Ia) and (Ib) that are potent modulators of the Wnt/β-catenin pathway. Provided are potent compounds that inhibit CREB binding protein (CBP)/β-catenin mediated signaling, compositions and pharmaceutical compositions comprising these compounds, and the use of these compounds for the treatment of any aberrant CBP/β-catenin mediated signaling disease or disorder, including but not limited to fibrosis, cancer, neurological disorders, metabolic disorders (including diabetes and fatty liver disease, e.g., alcoholic (ALD) and non-alcoholic hepatic steatosis (ALD and NAFLD, respectively), and including non-alcoholic steatohepatitis (NASH)), skin conditions (e.g., dermatitis, psoriasis, alopecia, skin aging, etc.) aging, and optionally further including one or more of pulmonary hypertension, congestive heart failure, chronic kidney disease, renal fibrosis, cardiac fibrosis, polycystic ovary syndrome (PCOS), endometriosis, and/or systemic fibrosis/scleroderma. Both therapeutic, and cosmetic methods are provided.

Compounds described herein possess at least one property or characteristic that is of therapeutic relevance. Candidate inhibitors may be identified by using, for example, an art-accepted assay or model. The Example section described assay(s) that were used to determine the Wnt/catenin signaling/CBP/catenin signaling pathway modulatory activity of the compounds described herein. The skilled artisan is aware of other procedures, assay formats, and the like that can be employed to generate data and information useful to assess the Wnt/catenin signaling/CBP/catenin signaling pathway modulators described herein. Candidate inhibitors can be further evaluated by using techniques that provide data regarding characteristics of the modulators (e.g., pharmacokinetic parameters), which will be apparent to the skilled artisan.

Another aspect of the present invention provides the methods to synthesize compounds of formula (Ia) and (Ib) as further detailed in the Example section. In general, the 1, 4-pyrazole containing compounds, IIc, of formula (Ia) and (Ib) can be readily prepared, according the following general synthetic scheme A, using the procedure of the copper-catalyzed sydnone-alkyne ligation in an organo-aqueous condition (see: e.g., Angew. Chem., Int. Ed. 2013, 52, 12056-12060). The alkyne IIa and the transformation of IIc to IId can be readily prepared based on the procedures disclosed in US 2021/0317123 A1. The sydnone lib is either commercially available or can be prepared by the methods known in the art (see: e.g., J. Med. Chem. 2002, 45 (24), 5397; CN 111057024A).

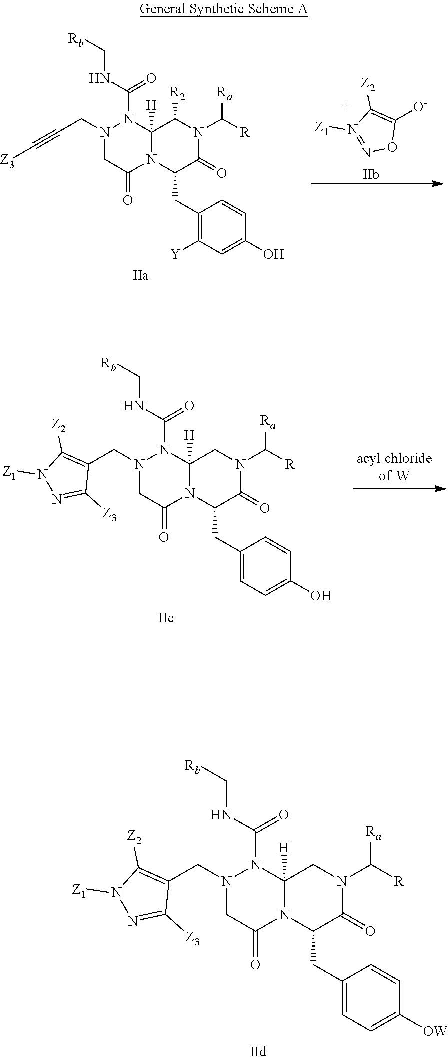

Compounds of formula (Ia) and (Ib) of the present invention may contain chiral centers and therefore may exist in different enantiomeric and diastereomeric forms. This invention relates to all optical isomers and all stereoisomers of compounds having the structures as defined above, both as racemic mixtures and as individual enantiomers and diastereoisomers of such compounds, and mixtures thereof, and to all pharmaceutical compositions and methods of treatment defined below that contain or employ them, respectively. In some embodiments, the compounds are the (S)-enantiomer. In other embodiments, the compounds are the (R)-enantiomer.

As the compounds of the present invention may possess at least two asymmetric centers, they are capable of occurring in various stereoisomeric forms or configurations. Hence, the compounds can exist in separated (+)- and (−)-optically active forms, as well as mixtures thereof. The present invention includes all such forms within its scope. Individual isomers can be obtained by known methods, such as optical resolution, optically selective reaction, or chromatographic separation in the preparation of the final product or its intermediate.

This invention also provides pharmaceutical compositions and formulations comprising one or more of the disclosed compounds in an amount sufficient, when administered to a warm-blooded mammalian subject having a disease or disorder mediated by aberrant CREB binding protein (CBP)/β-catenin signaling, to specifically inhibit the CBP/catenin mediated signaling within a warm-blooded mammalian subject. The amount of the administered compound preferably comprises a therapeutically effective amount, and in such cases the pharmaceutical compositions and formulations may comprise a therapeutically effective amount of the compound having the structure disclosed herein or a therapeutically acceptable salt thereof and a pharmaceutically acceptable carrier, diluent, or excipient therefor. All of these forms are encompassed within the scope of the present invention.

### Definitions

“Lower”, unless indicated otherwise, means that the number of the carbon atoms constituting the given radicals is between one and six.

“Halogen” means fluorine, chlorine, bromine or iodine.

“Halo” means fluoro, chloro, bromo or iodo.

“Alkyl” means a linear or branched, saturated, aliphatic radical having a chain of carbon atoms.

“Alkenyl” means a linear or branched, carbon chain that contains at least one carbon-carbon double bond.

“Alkynyl” means a linear or branched, carbon chain that contains at least one carbon-carbon triple bond.

“Alkylene”, unless indicated otherwise, means a linear or branched, saturated, aliphatic, polyvalent carbon chain.

“Oxy” means the radical —O—. It is noted that the oxy radical may be further substituted with a variety of substituents to form different oxy groups including hydroxy, alkoxy, aryloxy, heteroaryloxy and the like.

“Phosphate” or “Phosphate salt” means PO₃H₂, or PO₃⁻ and an appropriate counterion(s) (e.g., 1-2Na⁺, 1-2K⁺, or Ca⁺⁺, etc.), respectively.

“Thio” means the radical —S—. It is noted that the thio radical may be further substituted with a variety of substituents to form different thio groups including mercapto, alkylthio, arylthio, heteroarylthio and the like.

“Sulfinyl” means the radical —SO—. It is noted that the sulfinyl radical may be further substituted with a variety of substituents to form different sulfinyl groups including alkylsulfinyl, arylsulfinyl, heteroarylsulfinyl and the like.

“Sulfonyl” means the radical —SO₂—. It is noted that the sulfonyl radical may be further substituted with a variety of substituents to form different sulfonyl groups including alkylsulfonyl, arysulfonyl, heteroarylsulfonyl and the like.

“Alkoxy” means an oxygen moiety having a further alkyl substituent.

“Heteroatom” refers to an atom that is not a carbon atom and hydrogen atom. Particular examples of heteroatoms include, but are not limited to nitrogen, oxygen, and sulfur.

“Aryl” means a monocyclic or polycyclic radical wherein each ring is aromatic or when fused with one or more rings forms an aromatic ring.

“Heteroaryl” means a monocyclic or polycyclic aromatic radical wherein at least one ring atom is a heteroatom and the remaining ring atoms are carbon. “Cycloalkyl” means a non-aromatic, saturated or partially unsaturated, monocyclic, fused bicyclic or bridged polycyclic ring radical.

“Heterocycloalkyl” means cycloalkyl, as defined in this Application, provided that one or more of the atoms forming the ring is a heteroatom selected, independently from N, O, or S.

“Fused ring” as used herein refers to a ring that is bonded to another ring to form a compound having a bicyclic structure when the ring atoms that are common to both rings are directly bound to each other.

“Bridging ring” as used herein refers to a ring that is bonded to another ring to form a compound having a bicyclic structure where two ring atoms that are common to both rings are not directly bound to each other.

“Protected derivatives” means derivatives of compound in which a reactive site or sites are blocked with protecting groups. A comprehensive list of suitable protecting groups can be found in T. W. Greene, Protecting Groups in Organic Synthesis, 3rd edition, John Wiley & Sons, Inc. 1999.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality. A mixture of the two enantiomeric forms is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Animal” includes humans, non-human mammals (e.g., mice, rats, dogs, cats, rabbits, cattle, horses, sheep, goats, swine, deer, and the like) and non-mammals (e.g., birds, and the like).

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salt” or “salt” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts, which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Amount effective to treat” or “therapeutically effective amount” means that amount which, when administered to an animal for treating a disease, is sufficient to effect such treatment for the disease.

“Amount effective to prevent” means that amount which, when administered to an animal for preventing a disease, is sufficient to effect such prophylaxis for the disease.

“Treatment” or “treat” means any administration of a compound of the present invention and includes: (i) preventing the disease from occurring in an animal which may be predisposed to the disease but does not yet experience or display the pathology or symptomatology of the disease; (ii) inhibiting the disease in an animal that is experiencing or displaying the pathology or symptomatology of the diseased (i.e., arresting further development of the pathology and/or symptomatology); or (iii) ameliorating the disease in an animal that is experiencing or displaying the pathology or symptomatology of the diseased (i.e., reversing the pathology and/or symptomatology).

A “cosmeceutically effective amount” is amount which, when administered (e.g., transdermal, topically) is sufficient to affect cosmetic treatment of a cosmetic condition (e.g., wrinkles, hyperpigmentation, redness, rosacea, dryness, cracking, loss of vibrance, loss of elasticity, thinning, loss of vibrance, scarring, acne, sun damage, hair loss, loss of hair coloration, reduced cuticle growth, reduced nail growth).

### Therapeutic Uses and Pharmaceutical Compositions of Compounds of the Present Invention

According to aspects of present invention, exemplary diseases and conditions as discussed below, may be treated by modulating the common pathway (WNT-β-catenin signaling) shared by the disease states, regardless of cause or of tissue in which it is manifested. For example, treating a disease or disorder may comprise administering to a patient or a warm-blooded mammal, having a disease or disorder mediated by aberrant CREB binding protein (CBP)/β-catenin signaling, an amount of a compound of the present invention sufficient to inhibit CBP/catenin signaling and/or enhance p300/catenin mediated signaling.

The WNT/β-catenin pathway plays a crucial role in a broad array of diseases. Given the crucial role of WNT signaling in virtually every organ system in normal homeostasis and repair after injury, it is not surprising that aberrant regulation of this signaling cascade is associated with an array of diseases (see, e.g., Kahn, M., NATURE REVIEWS|DRUG DISCOVERY VOLUME 13|July 2014| 513). Beyond having an unequivocal role in multiple malignancies, aberrant WNT signaling is implicated as an important part in various other diseases, including neurological diseases, inflammatory and fibrotic disease, and disorders of endocrine function and bone metabolism in adults.

In particular aspects, aberrant WNT signaling is implicated in: cancer (e.g., cancer stem cell involvement in minimal residual disease in both solid (e.g., colon, pancreas, lung, liver, bladder, prostate, melanoma, glioma, meduloblastoma, osteosarcoma, uterine, endometrium and breast etc.) and liquid tumors (e.g., CML, CLL, AML, ALL etc.); in multiple myeloma; autoimmune disorders including Type I diabetes, rheumatoid arthritis, inflammatory bowel disease; topical disorders (e.g., psoriasis, vitiligo and atopic dermatitis); fibrosis including cardiac, liver, lung, kidney systemic and peritoneum (endometriosis) and ocular fibrosis; osteoarthritis and osteoporosis; metabolic diseases including Type II diabetes; hypertension; familial adenomatous polyposis; myelodysplastic syndrome (MDS); myeloproliferative proliferative neoplasms (MPN); CHIP (clonal hematopoiesis of indeterminate potential); pre-fibrotic conditions (e.g., AFLD, NASH, NAFLD, cirrhosis); polycystic kidney disease; polycystic ovary syndrome (PCOS), endometriosis, metabolic diseases; type II diabetes; hypertension; lung disorders (e.g., asthma and COPD); neurological diseases including, but not limited to neurodevelopment, autism, spectrum disorders, schizophrenia, and neurodegenerative disease including ALS, Huntington's, Parkinson's, Alzheimer's, frontotemporal dementia, multiple sclerosis (MS); and even more generally aging. The involvement of WNT signaling in particular representative diseases is outlined in more detail below.

Cancer. Aberrant regulation of WNT signaling has emerged as a recurrent theme in cancer biology. Constituents of WNT signaling can basically be characterized as either positively or negatively acting components, where the negatively acting components that principally act to suppress tumorigenesis are found mutated or in a loss-of-function status in cancer, whereas the positive components are activated. The discovery in 1991 that mutations in the tumor suppressor APC were associated with the vast majority of sporadic colorectal cancers via aberrant activation of WNT signaling provided considerable impetus to attempt to therapeutically target this pathway. Germ line defects in APC cause familial adenomatous polyposis, in which affected individuals develop hundreds of polyps in the large intestine at an early age and ultimately progress to colorectal cancer with 100% penetrance. Loss of function in both alleles of APC is required for tumorigenesis and is linked to the protein's ability to regulate β-catenin protein stability as well as chromosomal stability. APC is now noted as the most frequently mutated gene overall in human cancers. Mutations affecting the WNT pathway are not limited to colon cancer. For example, loss-of-function mutations in AXIN have been found in hepatocellular carcinomas, and oncogenic β-catenin mutations that were first described in colon cancer and melanoma were subsequently found to occur in a variety of solid tumors, including hepatocellular carcinomas, thyroid tumors and ovarian endometrioid adenocarcinomas. Epigenetic silencing is also frequently observed to alter levels of expression of negative regulators of the WNT-β-catenin pathway. For example, methylation of genes that encode putative extracellular WNT antagonists, such as the secreted Frizzled-related proteins (SFRPs), has been described in colon, breast, prostate, lung and other cancers. Increased expression of WNT ligands or effector proteins, including Dishevelled (DVL), has also been described. In particular, aberrant WNT signaling is implicated in cancer stem cells involved with minimal residual disease and relapse in both solid (e.g., colon, pancreas, lung, liver, bladder, prostate, melanoma, glioma, meduloblastoma, osteosarcoma, uterine, endometrium and breast etc.) and liquid tumors (e.g., CML, CLL, AML, ALL etc.). Specific small molecule CBP/β-catenin antagonists are effective in eliminating cancer stem cells, minimal residual disease and disease relapse (e.g. Kim, et al. Exp. Hematol. 2017 doi: 10.1016/j.exphem.2017.04.010). Furthermore, pre-malignancies and syndromes such as clonal hematopoiesis of indeterminate potential (CHIP), myelodysplastic syndrome (MDS), myelofibrosis (MF) and myeloproliferative neoplasms (MPN), Barrett's Esophagus, which are driven by defective stem cells, may be prophylactically eliminated by specific small molecule CBP/β-catenin antagonists (Thomas and Kahn Cell Biol. Toxicol. 2016 doi: 10:1007/s10565-016-9318-0).

Fibrosis. Fibrosis is characterized by an excessive accumulation of extracellular matrix components, which disrupts the physiological tissue architecture, leading to the dysfunction of the affected organ Fibrosis in general has been suggested to account for approximately 45% of deaths in industrialized countries, thereby highlighting the great medical need for effective antifibrotic therapies. Activated WNT/β-catenin signaling has been implicated in fibrosis in a number of organ systems, including the lungs, which indicates that this developmental pathway can be reactivated in adult tissues following injury. Specific small-molecule WNT modulation in several murine models of fibrosis (such as lung and kidney models) has proven to be extremely effective. Specific inhibition of the CBP/β-catenin interaction was shown to not only ameliorate but also to reverse late-stage fibrotic injury in murine models of lung, kidney, liver, cardiac, systemic fibrosis and endometriosis (Sci Rep. 2019 Dec. 27; 9(1):20056. doi: 10.1038/s41598-019-56302-4; Akcora et al., Biochim Biophys Acta Mol Basis Dis. 2018 March; 1864(3):804-818. doi: 10.1016/j.bbadis.2017.12.001; Xiao et al., Biochim Biophys Acta Mol Basis Dis. 2019 Jun. 1; 1865(6):1313-1322. doi: 10.1016/j.bbadis.2019.01.027. Epub 2019 Jan. 30; Zhao et al., Sci Rep. 2018 Jun. 12; 8(1):8996. doi: 10.1038/s41598-018-27064-2; Kimura et al., EBioMedicine 2017 September; 23:79-87. doi: 10.1016/j.ebiom.2017.08.016. Epub 2017 Aug. 19. Safety, Tolerability, and Preliminary Efficacy of the Anti-Fibrotic Small Molecule PRI-724, a CBP/β-Catenin Inhibitor, in Patients with Hepatitis C Virus-related Cirrhosis: A Single-Center, Open-Label, Dose Escalation Phase 1 Trial). In this trial the CBP/β-catenin antagonist PRI-724 administered by intravenous injection to patients with HCV cirrhosis at doses of 10 and 40 mg/m²/day for 12 weeks (1 week on and 1 week off) appeared to be safe, provided dose-dependent plasma exposure of the drug, and resulted in an improvement in liver histology and Child Pugh scores in several patients.

Pulmonary fibrosis. Pulmonary fibrosis destroys the lung's ability to transport oxygen and other gases into or out of the blood. This disease modifies the delicate and elastic tissues of the lung, changing these tissues into thicker, stiff fibrous tissue. This change or replacement of the original tissue is similar to the permanent scarring that can occur to other damaged tissues. Scarring of the lung reduces the lung's ability to allow gases (i.e. oxygen, carbon dioxide) to pass into or out of the blood. Gradually, the air sacs of the lungs become replaced by fibrotic tissue. When the scar forms, the tissue becomes thicker causing an irreversible loss of the tissue's ability to transfer oxygen into the bloodstream. Symptoms include shortness of breath, particularly with exertion; chronic dry, hacking cough; fatigue and weakness; discomfort in the chest; loss of appetite; and rapid weight loss. Several causes of pulmonary fibrosis are known and they include occupational and environmental exposures. Many jobs, particularly those that involve mining or that expose workers to asbestos or metal dusts, can cause pulmonary fibrosis. Workers doing these kinds of jobs may inhale small particles (like silica dusts or asbestos fibers) that can damage the lungs, especially the small airways and air sacs, and cause the scarring associated with fibrosis. Agricultural workers also can be affected. Some organic substances, such as moldy hay, cause an allergic reaction in the lung. This reaction is called Farmer's Lung and can cause pulmonary fibrosis. Other fumes found on farms are directly toxic to the lungs. Another cause is Sarcoidosis, a disease characterized by the formation of granulomas (areas of inflammatory cells), which can attack any area of the body but most frequently affects the lungs. Certain medicines may have the undesirable side effect of causing pulmonary fibrosis, as can radiation, such as treatment for breast cancer. Connective tissue or collagen diseases such as systemic sclerosis are also associated with pulmonary fibrosis. Although genetic and familial factors may be involved, this cause is not as common as the other causes listed above. In Chronic Obstructive Pulmonary Disease (COPD), connective tissue proliferation and fibrosis can characterize severe COPD. COPD can develop as a result of smoking or chronic asthma.

Idiopathic Pulmonary Fibrosis (IPF). When all known causes of interstitial lung disease have been ruled out, the condition is called “idiopathic” (of unknown origin) pulmonary fibrosis (IPF). Over 83,000 Americans are living with IPF, and more than 31,000 new cases develop each year. This debilitating condition involves scaring of the lungs. The lungs' air sacs develop scar, or fibrotic tissue, which gradually interferes with the body's ability to transfer the oxygen into the bloodstream, preventing vital organs and tissue from obtaining enough oxygen to function normally. There are several potential causes of IPF, including viral illness e.g., SARS-CoV-2 and allergic or environmental exposure (including tobacco smoke). There is also a familial form of the disease, known as familial idiopathic pulmonary fibrosis. Patients with IPF suffer similar symptoms to those with pulmonary fibrosis when their lungs lose the ability to transfer oxygen into the bloodstream. The symptoms include shortness of breath, particularly during or after physical activity; spasmodic, dry cough; gradual, unintended weight loss; fatigue and weakness; chest discomfort; clubbing, or enlargement of the ends of the fingers (or sometimes the toes) due to a buildup of tissue. These symptoms can greatly reduce IPF patients' quality of life. Pulmonary rehabilitation, and oxygen therapy can reduce the lifestyle-altering effects of IPF, but do not provide a cure.

Diabetes and Metabolic Diseases. Wnt signaling is critically important not only in stem cell maintenance, differentiation, and migration, but also in organogenesis. WNT signaling also has crucial roles in various endocrine functions and has therefore been implicated in several endocrine disorders. WNT signaling is important in the regulation of insulin sensitivity and its dysregulation is implicated in the development of diabetes. In particular, WNT10B increases insulin sensitivity in skeletal muscle cells. Overexpression of WNT5B induces adipogenesis. Decreased expression of β-catenin-independent WNT5B, which has been demonstrated in patients with type 2 diabetes, may increase susceptibility to type 2 diabetes. β-catenin/TCF7L2-dependent Wnt signaling (the canonical pathway) is involved in pancreas development, islet function, and insulin production and secretion. Glucagon-like peptide-1 (GLP-1) and the chemokine stromal cell-derived factor-1 (SDF1) modulate canonical Wnt signaling Additionally, polymorphisms in the transcription factor TCF7L2 (also known as TCF4), are linked to increased susceptibility to type 2 diabetes. Individuals with at-risk alleles of TCF7L2 exhibit impaired insulin secretion, and TCF7L2 in pancreatic β-cells appears to have a crucial role in glucose metabolism through the regulation of pancreatic β-cell mass.

Experimental loss of TCF7L2 function in islets impairs glucose-stimulated insulin secretion, suggesting that perturbations in the Wnt signaling pathway may contribute substantially to the susceptibility for, and pathogenesis of, T2D. Interestingly, nicotine has been shown to enhance renal cell proliferation and fibronectin production under high glucose partly via activating the Wnt/β-catenin pathway. Increased Wnt/CBP/β-catenin signaling may initially induce pancreatic β-cell expansion; however, continuous Wnt driven mitogenic signaling may eventually lead to a loss of differentiation capacity and functionality.

Recently, researchers treated cadaver-derived intact human islets with conditioned medium from L-cells that constitutively produced WNT3A, R-spondin 3 and Noggin, to which inhibitors of RHO-associated protein kinase (ROCK) and RHOA were added to augment cell survival. This led to an approximately 20-fold increase in β-cell proliferation compared with glucose alone. Importantly, treatment with this conditioned medium did not impair glucose-stimulated insulin secretion or decrease the insulin content of the cells. In transcriptome-wide gene expression profiling and follow-up signaling studies, the researchers showed that the conditioned media treatment specifically promoted WNT signaling.

Neurological diseases. The importance of WNT signaling during embryonic development of the central nervous system is well established. The WNT pathway also regulates nervous system patterning and the regulation of neural plasticity. WNTs also have a role in axon guidance as well as in influencing synapse formation. Therefore, it is not surprising that aberrations in WNT signaling have been observed in neurological diseases in adulthood. For example, a Scottish family with a high incidence of schizophrenia, depression and bipolar disorder was found to carry a balanced chromosomal translocation involving the gene DISC1 (disrupted in schizophrenia 1). Subsequently, the protein product of DISC1 was found to have an important role in neural development and neural progenitor proliferation. DISC1 directly interacts with and inhibits GSK33 activity, thereby enhancing β-catenin-mediated transcription.

Neuroanatomical observations and functional magnetic resonance imaging (MRI) have indicated that a major pathological hallmark in autistic individuals may be a premature overgrowth of the cerebral cortex, hippocampus, amygdala and cerebellum. Interestingly, transgenic mice expressing a constitutively active form of β-catenin in neuronal precursor cells developed a grossly enlarged cerebral cortex, hippocampus and amygdala. Importantly, microdeletion and microduplication copy number variations of genes involved in the canonical WNT signaling pathway (for example, Frizzled 9 (FZD9), B cell lymphoma 9 (BCL9) or cadherin 8 (CDH8)) are found in patients with autism spectrum disorder. Association studies investigating WNT2, DISC1, MET, dedicator of cytokinesis protein 4 (DOCK4) or Abelson helper integration site 1 (AHI1; also known as jouberin) have provided additional evidence that the canonical WNT pathway might be affected in autism.

The WNT signaling cascade has also been implicated in Alzheimer's disease. Presenilin proteins, which have been associated with early-onset Alzheimer's disease, are negative regulators of canonical WNT signaling. Variant alleles in the WNT receptor LRP6 (low-density lipoprotein receptor-related protein 6) have been associated with Alzheimer's disease in population-based linkage analyses. This suggests that multiple mechanisms leading to aberrant WNT-mediated regulation of adult neurogenesis may be associated with Alzheimer's disease. As the underlying cause (or causes) of Alzheimer's disease have not been clearly elucidated, the mechanisms whereby aberrant WNT regulation may have a role in Alzheimer's disease are also not known. WNT signaling is involved in brain vascularization and blood-brain barrier formation, in synaptogenesis, in amyloid-β-induced neuroinflammation and neurotoxicity, as well as in neuronal degeneration. Aberrant regulation of any or all of these processes could contribute to disease initiation and progression.

Skin. The WNT signaling cascade has also been implicated in skin development and maintenance (see, e.g., STEM CELLS 2018; 36:22-35). Secreted Wnt proteins can stimulate multiple intracellular signaling pathways and act as growth factors that regulate diverse processes, including cell proliferation, differentiation, migration, and polarity. Among Wnt stimulated pathways, Wnt/β-catenin signaling is known as an important regulatory pathway that governs developmental processes and fate choices during tissue morphogenesis. Wnt signaling is one of the major cues directing skin development and maintenance. Although Wnt signaling has been mainly implicated in HF (Hair Follicle) induction during skin development, it has also been recently shown to regulate epidermal stratification. In primary human keratinocytes, Wnt5a acts as an autocrine stimulus to promote extracellular calcium-induced keratinocyte differentiation by coupling with Wnt/β-catenin pathway. Throughout life the skin epidermis is regularly renewed. Skin epidermal SCs, capable of self-renewal and differentiation, provide unlimited sources of cells to maintain tissue homeostasis, as well as to regenerate HFs and repair the epidermis after injury. Wnt signaling is critical in all of these processes and Wnt dependent signaling plays crucial roles in the maintenance, activation, and fate determination of the SC populations.

### Vaccine (e.g., SARS-CoV-2 (COVID-19), Influenza, Etc.) Enhancement

Age. With age, the immune system loses some of its vigor (immunosenescence, reflected by fewer naïve T cells, and B cells), which is believed to contribute to the higher vulnerability to COVID-19 and more generally infection in older age subject groups (e.g., ≥60 yrs or ≥65 yrs, for humans). Additionally, vaccines may perform poorly in such older subject groups, which often experience chronic inflammation (inflammaging, characterized by impaired clearance of dead and dying cells from sites of immune activity, and high baseline serum concentrations of C reactive protein (CRP) and cytokines, e.g., interleukin-6 (IL-6), and IL-8)), which factors may inhibit antigen-specific (e.g., anti-viral) immunity, e.g., influenza virus (Willyard, C., Nature Vol. 586, 2020; Akbar & Gilroy, Science 369 (6501), 256-257, 2020, DOI: 10.1126/science.abb0762; A. Parmigiani et al., PLOS ONE 8, e79816 (2013)).

Current influenza vaccination strategies prioritizing elderly persons (55 to 75 yrs) have been found to be less effective than believed at reducing serious morbidity and mortality in this population, suggesting that supplementary strategies to boost the effectiveness of vaccination, essentially dependent on immune memory and recall, may be necessary (Anderson et al., Ann Intern Med. 2020; 172:445-452. doi:10.7326/M19-3075).

Likewise, SARS-CoV-2 causes severe respiratory disease (coronavirus disease 2019, COVID-19) that mostly induces mild to moderate symptoms in younger individuals, but induces devastating morbidity and mortality in older individuals. A key hallmark of severe disease is exuberant inflammation in the respiratory tract of patients (Merad & Martin, Nat. Rev. Immunol. 20, 355, 2020).

Memory T cells. A hallmark of the aging immune system is its failure to induce long-lived memory (Kim, Chulwoo, et al., Cell Reports 25, 2148-2162, Nov. 20, 2018). Moreover, enforcing asymmetric cell division (ACD) rates can improve long-term survival and function of T cells and open new perspectives for vaccination (Borsa, et al., Sci. Immunol. 4, eaav1730 (2019). It has been demonstrated that asymmetric cell division is responsible for the dichotomy between the generation of memory T cells and effector cells from a common precursor activated by antigenic recognition in the context of an antigen-presenting cell (Morrot, Alexandre, Ann Transl Med 2017; 5(5):121; citing Verbist K C, Guy C S, Milasta S, et al. Metabolic maintenance of cell asymmetry following division in activated T lymphocytes (Nature 2016; 532:389-93)). Moreover, memory T cells appear to use asymmetric cell division to generate cellular heterogeneity when faced with pathogen rechallenge (Ciocca, Maria, L. et al., The Journal of Immunology, 2012, 188: 4145-4148).

Currently suggested approaches to vaccine enhancement may involve the use of vaccine adjuvants, higher doses of viral antigen, or identification of drugs that might improve vaccine responses in older population group (e.g., rejuvenating the immune system). For example, mTOR inhibitors (Mannick, J. B. et al. Sci. Transl. Med. 10, 449, eaaq1564, 2020) (e.g., RTB101, rapamycin, metformin) have been proposed. Anti-inflammatory drugs (e.g., losmapimod, dexamethasone), and senolytics (e.g., fisetin) have also been proposed for boosting immunity.

There is, however, yet an urgent need, for more effective compositions and methods for enhancing vaccine (e.g., anti-viral vaccines for e.g., influenza, SARS, SARS-CoV-2, HPV, HEP-A, HEP-B, Herpes Zoster, etc.) response, for example, e.g., by; metabolic maintenance of cell asymmetry following division in activated T cells in the subject; and/or enhancing antigen-specific immunity by increasing the number and/or persistence of differentiated memory T-cells; and/or enhancing the presentation of antigens to T-cells by antigen presenting cells to enhance cooperativity between the innate and acquired immune systems (Ljungberg, Johanna K. et al, Front. of Immunol. 10; 2521, 2019), particularly in elderly subjects (e.g., 55-75; ≥60 yrs; ≥65 yrs).

CBP/Catenin inhibitors. PRI-724 (a specific CBP/β-catenin inhibitor) treatment of ART-suppressed SIVmac251-infected RMs has been shown to decrease proliferation of T memory stem cells (SCM) and central memory T-cells (CM) T-cells and modify the SCM and CM CD4+ T-cell transcriptome towards a profile of more differentiated memory T-cells, demonstrating that stemness pathways of long-lived memory CD4+ T-cells can be pharmacologically modulated in vivo, thus establishing a novel strategy to target HIV persistence (Mavigner, M. et al., J. Virol. doi:10.1128/JVI.01094-19).

According to particular aspects of the present invention, there is a need for safe and effective methods of treatment (e.g., prophylactic and/or therapeutic) to target fundamental ageing mechanisms at around the time of “vaccination”.

According to particular aspects of the present invention, the disclosed CBP/Catenin inhibitors have substantial utility for enhancing vaccination (e.g., anti-viral vaccines for e.g., influenza, SARS, SARS-CoV-2, HPV-A, HPV-B, Herpes Zoster etc.), particularly in aged individuals (e.g., 55-75; ≥60 yrs; ≥65 yrs), e.g., by: metabolic maintenance of cell asymmetry following division in activated T cells in the subject; and/or enhancing antigen-specific immunity by increasing the number and/or persistence of differentiated memory T-cells; and/or enhancing the presentation of antigens to T-cells by antigen presenting cells to enhance cooperativity between the innate and acquired immune systems, particularly in elderly subjects (e.g., 55-75; ≥60 yrs; ≥65 yrs). According to further aspects, the compounds may be administered prophylactically and/or therapeutically, including administration as a primer before vaccination, and/or co-administration with vaccination, and/or administration or co-administration (e.g., along with a vaccine booster) subsequent to initial vaccination.

According to further aspects, the compounds may be used to enhance vaccination in mammalian (e.g., human) subjects with any vaccine.

### SARS-CoV-2 (COVID-19) Tissue (Lung, Liver, Kidney, Heart, Etc.) Destruction (e.g., Pulmonary Fibrosis, ARDS)

As discussed above, inflammaging has many implications for COVID-19 patients (e.g., as discussed in Akbar & Gilroy, supra). While accumulation of senescent cells in the respiratory tract of older patients may be involved in the initiation of an inflammatory cascade that could inhibit T cell responses to virally infected cells that are present, high amounts of inflammation alone do not explain the devastating tissue destruction that is observed in the lungs of COVID-19 patients with severe disease, and it may be that age-associated changes in T cells have a role in the immunopathology. T lymphocytes that are highly differentiated and exhibit senescence-like characteristics accumulate in older individuals. While these aged T cells lose the capacity to proliferate after activation and express multiple markers of senescence, including DNA damage associated proteins [e.g., phosphorylated histone H2AX (gH2AX)] and cyclin-dependent kinase inhibitors (e.g., p16INK4A), they are nonetheless highly efficient cytotoxic cells, express NKRs, and can kill different cell types that express NKR ligands, including senescent non-lymphoid cells. Another consequence of the inflammation is the induction of NKR ligand expression by cells in the lung that would make them susceptible to killing by infiltrating T cells that express NKRs (Id).

Lung Fibrosis. Almost all COVID-19-related serious consequences feature pneumonia, and many have ground glass opacities, etc., many (about 40%) developing acute respiratory distress syndrome (ARDS). There is a concern that some organs, including the lungs, might have long-term impairment following infection, inflammation and lack of resolution (e.g., pulmonary fibrosis, which is a recognized sequelae of ARDS). Mechanical ventilation is the most important supportive therapy for patients with ARDS, including COVID-19 patients, but it can induce or aggravate lung injury, referred to as ventilator-induced-lung-injury (VILI) (Slutsky and Ranieri NEJM 2013). Although the virus is eradicated in patients who have recovered from COVID-19, the removal of the cause of lung damage does not, in itself, preclude the development of progressive, fibrotic irreversible interstitial lung disease. Furthermore, even a relatively small degree of residual but non-progressive fibrosis could result in considerable morbidity and mortality in an older population of patients who had COVID-19, many of whom will have pre-existing pulmonary conditions (Bem, Reinout A.; https://doi.org/10.1016/S2213-2600(20)30222-8). The description of the group in whom SARS-CoV-2 infection is most lethal is also highly representative of patients suffering with idiopathic pulmonary fibrosis (IPF). Anti-fibrotic therapies that are available or in development could have value in preventing severe COVID-19 in patients with IPF, have the potential to treat severe COVID-19 in patients without IPF, and might have a role in preventing fibrosis after SARS-CoV-2 infection (George et al, Lancet August 2020).

Acute lung injury and ARDS are the major cause of mortality in COVID-19. While conventional therapy may be possible with such agents as pirfenidone and nintedanib, pirfenidone and nintedanib are currently commercially available only in oral form and so cannot be used in patients who are intubated and mechanically ventilated, thereby restricting their use in those individuals with severe COVID-19 on the intensive care unit (ICU). Further, pirfenidone should be avoided if patients have an estimated glomerular filtration rate of less than 30 mL/min per 1.73 m². Moreover, both pirfenidone and nintedanib can be associated with hepatotoxicity, and liver dysfunction is common in patients infected with SARS-CoV-2. A further uncertainty relates to the rapidity (rate) with which antifibrotic agents act, where known agents may have little value in ventilated patients where the opportunity for effective treatment has already passed (Id).

According to particular aspects of the present invention, there is a need for safe and effective methods of treating (e.g., prophylactic and/or therapeutic) SARS-CoV-2 (COVID-19) lung tissue destruction (e.g., pulmonary fibrosis, ARDS).

As summarized above under “THERAPEUTIC USES AND PHARMACEUTICAL COMPOSITIONS OF THE PRESENT INVENTION” (pg. 29-34), the utility of CBP/β-Catenin inhibitors for treating aspects of fibrosis has been recognized.

According to particular aspects of the present invention, therefore, the disclosed CBP/Catenin inhibitors have substantial utility, particularly in aged individuals (e.g., 55-75; ≥60 yrs; ≥65 yrs), for treating SARS-CoV-2 (COVID-19) tissue (e.g., lung, liver, etc.) destruction (e.g., pulmonary fibrosis, ARDS), including both in the acute phase of the illness and in preventing long-term complications. According to further aspects, the compounds may be administered prophylactically and/or therapeutically, in either case preferably initiated before or within the first 1-3 weeks, preferably initiated before or within the first week, of ARDS onset.

### Administration:

The pharmaceutical composition of the present invention is formulated to be compatible with its intended route of administration. Examples of routes of administration include parenteral, e.g., oral (e.g., in capsules or tablets), intravenous, intradermal, subcutaneous, inhalation, transdermal (topical), transmucosal, and rectal administration. Solutions or suspensions (e.g., injection) used for parenteral (particularly, intravenous), intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In addition, pH may be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound, e.g., a compound having general formula (Ia) in the required amount, in an appropriate solvent with one or a combination of ingredients enumerated above, as required, followed by filtered sterilization. Generally, dispersions are prepared by incorporating the active compound into a sterile vehicle that contains a dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent I such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In particular embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers.

It can be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. For instance, in certain embodiments, a pharmaceutical composition of the present invention is one suitable for oral administration in unit dosage form such as a tablet or capsule that contains from about 1 mg to about 10 g of the compound of this invention. In some other embodiments, a pharmaceutical composition of the present invention is one suitable for intravenous, subcutaneous or intramuscular injection. A patient may receive, for example, an intravenous, subcutaneous or intramuscular dose of about 1 μg/kg to about 1 g/kg of the compound of the present invention. The intravenous, subcutaneous and intramuscular dose may be given by means of a bolus injection or by continuous infusion over a period of time. Alternatively a patient will receive a daily oral dose approximately equivalent to the daily parenteral dose, the composition being administered 1 to 4 times per day.

The compounds may be administered intravenously (e.g., by continuous drip infusion or rapid intravenous administration) to mammals inclusive of human. In such case, the dose may be selected appropriately depending on various factors such as the body weight and/or age of patients, and/or the degree of the symptom and an administration route. For example, the dose of the compound for intravenous administration is generally in the range of 1 to 10000 mg/day/m² human body surface area, preferably in the range of 1 to 5000 mg/day/m² human body surface area, and more preferably 10 to 5000 mg/day/m² human body surface area by continuous drip infusion administration.

These therapeutic agents may be administered according to how often per day (one or more times per 24 hour period), including the time between doses (e.g. every 6 hours), the times when the doses are to be administered (e.g. at 8 a.m. and 4 p.m. daily) and the amount of the therapeutic agent (e.g. number of capsules) to be given at a specific time.

### WORKING EXAMPLES

An illustration of the preparation of exemplary compounds of the present invention is shown in the representative examples and schemes below, wherein specific non-limiting working Examples of compounds are intended to illustrate particular exemplary embodiments of the present invention, and are not intended to limit the scope of the specification or the claims in any way. The compounds of the present invention may be prepared by the synthetic sequences shown in the non-limiting Examples and Schemes below. A skilled artisan will appreciate that other routes of synthesis may be employed as well. In particular, other routes of synthesis may in fact be applied to certain aspects of the present invention. The skilled artisan is referred to general textbooks, such as March's Advanced Organic Chemistry (Michael B. Smith & Jerry March, Wiley-Interscience, 2000), The Practice of Medicinal Chemistry (Camile G. Wermuth, Academia Press, 2003) and Protective Groups in Organic Synthesis (Theosora W. Greene & Peter G. M. Wuts; John Wiley & Sons Inc, 1999), all incorporated by reference herein for their respective teachings.

### Example 1

### Reagents, Synthetic Methods, and Biological Characterization Assay Used

Unless otherwise noted, all reagents, starting materials and solvents were obtained from commercial suppliers and used without further purification. Concentration or evaporation refers to evaporation under vacuum using a Buchi rotatory evaporator, and/or followed by evaporation to dryness under high vacuum. Reaction products were purified by silica-gel chromatography with the solvent system indicated, or by HPLC purification using a C18 reverse phase semi-preparative HPLC column with solvent A (0.1% of TFA in water) and solvent B (0.1% of TFA in CH₃CN) as eluents. All final products have at least 95% purity as determined by analytical HPLC analysis with UV detection at 210 nm and/or 254 nm. Reported yields are isolated yields.

Analytical HPLC analysis was performed on an Agilent 1100 HPLC with a Phenomenex Luna C18 (2) column (3 micron, 150×4.6 mm id) at a flow rate of 0.6 mL/min, eluting with a binary solvent system A and B using a 10%-90% B in 20 min and then 90%-95% B in 5 min gradient elution (gradient elution 1), or a 70%-95% B in 25 min and then 95%-100% B in 3 min gradient elution (gradient elution 2) (A: Milli-Q water with 0.1% TFA; B: CH₃CN with 0.1% TFA) with initial operating pressure in the range of 120 to 140 bar. NMR spectra were recorded on a Bruker AV-300 or AV-301 300 MHz NMR instrument using DMSO-d₆ or CDCl₃ with TMS as an internal standard. Mass spectra data was obtained with Bruker Esquire Liquid Chromatography-Ion Trap Mass Spectrometer.

The following abbreviations are used in the synthetic examples: aq (aqueous), h (hour), min (minutes), sat'd (saturated), THF (tetrahydrofuran), rt (room temperature), Et₃N (triethylamine), NaCl (sodium chloride), MgSO₄ (magnesium sulfate), CDCl₃ (deuterated chloroform), H₂O (water), HCl (hydrochloric acid), MeOH (methanol), NaOH (sodium hydroxide), TFA (trifluoroacetic acid), Na₂CO₃ (sodium carbonate), CH₂Cl₂ (methylene chloride), EtOAC (ethyl acetate), DMF (dimethylformamide), EtOH (ethanol), DMSO (dimethyl sulfoxide), DMSO-d₆ (dimethyl sulfoxide-de), NaHCO₃ (sodium bicarbonate), HPLC (high performance liquid chromatography), ESI-MS or MS (ESI) (electrospray ionization-mass spectrometry), NMR (nuclear magnetic resonance), DIEA (diisopropylethylamine), sta'd NaCl or brine (saturated aqueous NaCl solution), NBS (N-bromosuccinimide), HEDTA (N-(2-hydroxyethyl)ethylenediamine-N,N′,N′-triacetic acid), and other similar standard abbreviations are used herein.

Biological Characterization of Exemplary Compounds of the invention were performed in at least the following assays:

SuperTOPFLASH Cell-based Luciferase Assay. Hek-293, STF1.1 cells are maintained in DMEM, 10% FBS, Pen-Strep supplemented with 200 μg/mL G418. On the day prior to assay, cells are split into a white, opaque 96-well plate at 10,000 cells per well in 50 μL of complete medium without G418 (for screening of Wnt-signaling inhibitors, G418 can be left out during screening process). After allowing the cells to stabilize and attach overnight, 40 μL of complete medium (without G418) containing 2.5× final concentration of compound or DMSO control is added to the cells and allowed to incubate for 1 hour at 37° C., 5% CO₂ prior to adding 10 μL of a 100 mM LiCl solution prepared in complete medium (without G418). After 24 hours, 100 μL of BrightGlo (Promega, Cat. #: G7573) is added to each well and the plate is shaken for 5 minutes prior to reading on the Perkin-Elmer EnVision Plate Reader. For example, on the day prior to assay: cells are split into a white opaque 96-well plate at 10,000 cells per well in 50 μL of complete growth medium; the plate is incubated overnight at 37° C., 5% CO₂ and the cells allowed to attach; the next day inhibitors to be tested are prepared in complete growth medium at 2.5× the desired final concentration (all conditions are done in duplicates), and 40 μL of the medium containing the 2.5× concentration of compound is added to each well (include 2 wells for stimulation control, 2 wells for DMSO control, and wells for the positive control ICG-001 (e.g., 2, 5, and 10 micromolar)); once all inhibitors and controls are added, incubate the plate for 1 hour at 37° C., 5% CO₂ (while plate is incubating, prepare fresh 100 mM LiCl in complete growth medium); after 1 hour, the plate is removed from the incubator and 10 μL of the medium containing 100 mM LiCl are added to each well (except for the two wells of the unstimulated control, to which 50 microliters of just complete medium is added); the plate is incubated for 24 hours at 37° C., 5% CO₂; after 24 hours, 100 microliters of BrightGlo (Promega, Cat. #: G7573) is added to each well, the plate is shaken for 5 minutes to ensure complete lysis, and the plate is then read on a Perkin-Elmer EnVision 96-well plate reader.

### Example 2

### Synthesis of the Alkyne IIa

The key intermediates IIa (see “General Synthetic Scheme A” at page 15 herein) used for the preparation of exemplary compounds of the present invention were prepared according to the procedures disclosed in US 2021/0317123 A1. Both the key intermediates and the corresponding starting materials are listed in Table 1 below:

TABLE 1
Intermediates and starting materials used in preparing exemplary
compounds.

| | | MS (ESI) of IIa |
|---|---|---|
| key intermediate (IIa) | Starting material | [m/z (M + H)⁺] |
| 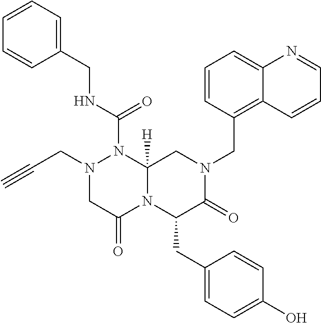 [SMILES: C#CCN1CC(=O)N2[C@@H](Cc3ccc(O)cc3)C(=O)N(Cc3cccc4ncccc34)C[C@@H]2N1C(=O)NCc1ccccc1] | 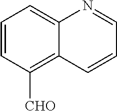 [SMILES: O=Cc1cccc2ncccc12] | 589.3 |
| 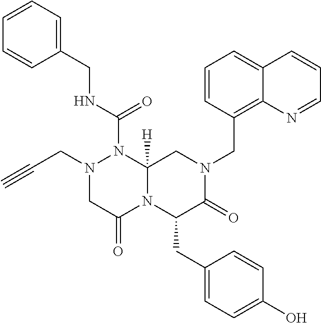 [SMILES: C#CCN1CC(=O)N2[C@@H](Cc3ccc(O)cc3)C(=O)N(Cc3cccc4cccnc34)C[C@@H]2N1C(=O)NCc1ccccc1] | 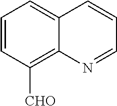 [SMILES: O=Cc1cccc2cccnc12] | 589.3 |
| 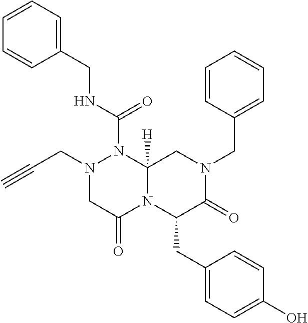 [SMILES: C#CCN1CC(=O)N2[C@@H](Cc3ccc(O)cc3)C(=O)N(Cc3ccccc3)C[C@@H]2N1C(=O)NCc1ccccc1] | 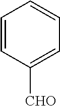 | 538.1 |
| 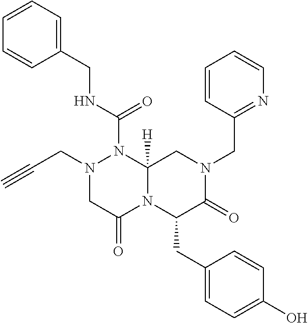 [SMILES: C#CCN1CC(=O)N2[C@@H](Cc3ccc(O)cc3)C(=O)N(Cc3ccccn3)C[C@@H]2N1C(=O)NCc1ccccc1] | 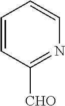 | 539.2 |
| 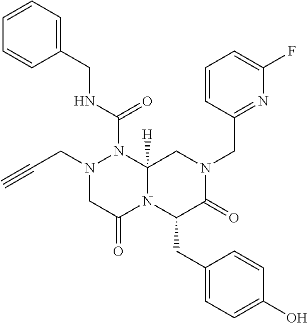 [SMILES: C#CCN1CC(=O)N2[C@@H](Cc3ccc(O)cc3)C(=O)N(Cc3cccc(F)n3)C[C@@H]2N1C(=O)NCc1ccccc1] | 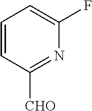 | 557.1 |
| 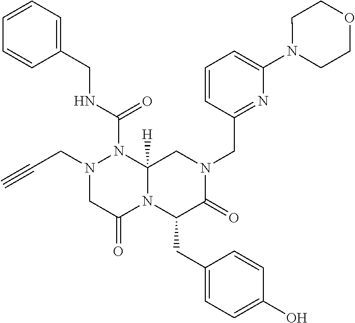 [SMILES: C#CCN1CC(=O)N2[C@@H](Cc3ccc(O)cc3)C(=O)N(Cc3cccc(N4CCOCC4)n3)C[C@@H]2N1C(=O)NCc1ccccc1] | 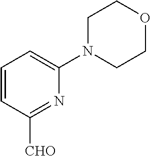 [SMILES: O=Cc1cccc(N2CCOCC2)n1] | 624.3 |
| 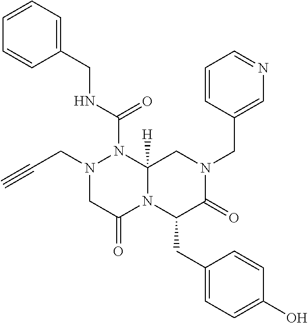 [SMILES: C#CCN1CC(=O)N2[C@@H](Cc3ccc(O)cc3)C(=O)N(Cc3cccnc3)C[C@@H]2N1C(=O)NCc1ccccc1] | 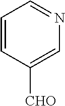 | 539.2 |

### Example 3

### Synthesis of (6S,9aS)—N-benzyl-6-(4-hydroxybenzyl)-4,7-dioxo-2-((1-phenyl-1H-pyrazol-4-yl)methyl)-8-(quinolin-5-ylmethyl)octahydro-1H-pyrazino[2,1-c][1,2,4]triazine-1-carboxamide (1)

Compound 1 was prepared according to Scheme 1, using the procedure analogous to that reported (see: Angew. Chem., Int. Ed. 2013, 52, 12056-12060).

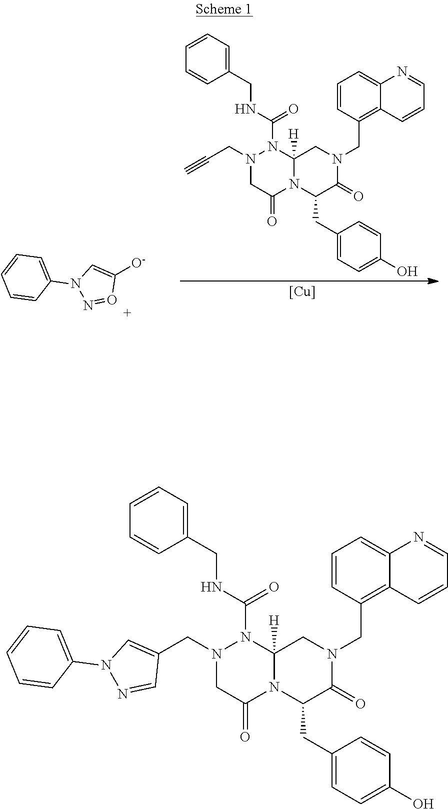

To a solution of 3-phenylsydnone (11.5 mg, 0.0707 mmol) in 1.3 mL of tert-butanol/water mixture (55:45) was added (6S,9aS)—N-benzyl-6-(4-hydroxybenzyl)-4,7-dioxo-2-(prop-2-ynyl)-8-(quinolin-5-ylmethyl)octahydro-1H-pyrazino[2,1-c][1,2,4]triazine-1-carboxamide (50 mg, 0.0707 mmol), sodium ascorbate (28 mg, 0.141 mmol), Copper(II) sulfate pentahydrate (3.5 mg, 0.0141 mmol), bathophenanthrolinedisulfonic acid disodium salt trihydrate (8.3 mg, 0.0141 mmol) and triethanolamine (10.6 mg, 0.0707 mmol). The reaction mixture was stirred at 60° C. for 20 hours, then quenched with 1.4 mL of HEDTA aqueous solution (50 mM) and poured into EtOAc (10 mL) and sat'd NaHCO₃ (10 mL). The aqueous layer was extracted with EtOAc (10 mL). The combined organic extracts were washed with sat'd NaCl (5 mL), dried (NaSO₄) and evaporated. Purification by silica-gel chromatography (CH₂Cl₂/CH₃OH/NH₄OH: 270:9:1 and 190:9:1) and followed by lyophilization with CH₃CN and Milli-Q water gave the title product (36.3 mg) as a white solid. MS (ESI): m/z 707.2 (M+H)⁺; analytical HPLC: 14.3 min (96% pure).

### Example 4

### Synthesis of (6S,9aS)—N-benzyl-2-((5-bromo-1-phenyl-1H-pyrazol-4-yl)methyl)-6-(4-hydroxybenzyl)-4,7-dioxo-8-(quinolin-5-ylmethyl)octahydro-1H-pyrazino[2,1-c][1,2,4]triazine-1-carboxamide (2)

Compound 2 was prepared according to Scheme 2, using the procedures reported set forth in steps 1-2 (see: Angew. Chem., Int. Ed. 2013, 52, 12056-12060; Org. Lett. 2015, 17, 362-365).

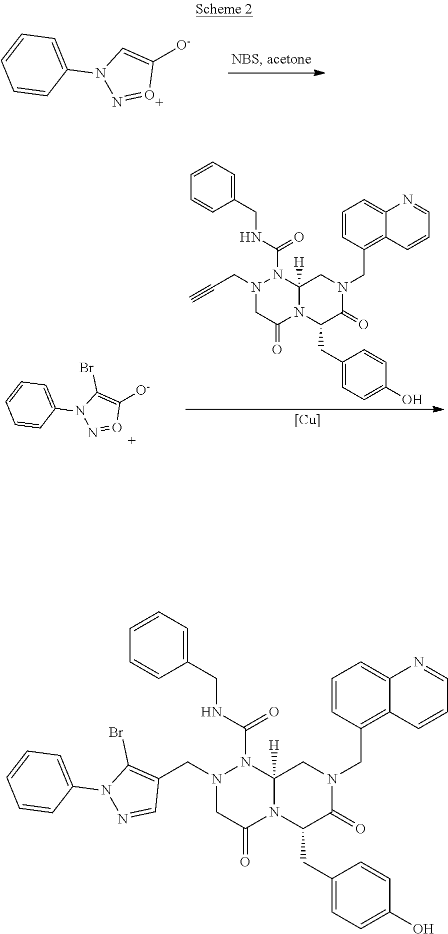

### Step 1: 3-phenyl-4-bromo-sydnone

To a solution of 3-phenylsydnone (11.5 mg, 0.0707 mmol) in 1 mL of acetone was added NBS (15.1 mg, 0.0848 mmol). The reaction mixture was stirred at room temperature overnight. Evaporation to dryness gave the title product as an off-white solid, which was used in the next step without further purification. MS (ESI): m/z 240.8 and 242.7 (M+H)⁺; analytical HPLC: 14.5 min.

### Step 2: (6S,9aS)—N-benzyl-2-((5-bromo-1-phenyl-1H-pyrazol-4-yl)methyl)-6-(4-hydroxybenzyl)-4,7-dioxo-8-(quinolin-5-ylmethyl)octahydro-1H-pyrazino[2,1-c][1,2,4]triazine-1-carboxamide

The title compound 2 as a white solid was obtained, according to the procedure described in Example 3. MS (ESI): m/z 785.2 and 787.1 (M+H)⁺; analytical HPLC: 14.9 min (97% pure).

### Example 5

### Synthesis of (6S,9aS)—N-benzyl-6-(4-hydroxybenzyl)-4,7-dioxo-2-((1-phenyl-1H-pyrazol-3-yl)methyl)-8-(quinolin-8-ylmethyl)octahydro-1H-pyrazino[2,1-c][1,2,4]triazine-1-carboxamide (3)

Compound 3 was prepared according to the procedures disclosed in US 2021/0317123 A1 set forth in steps 1-5 of Scheme 3 below.

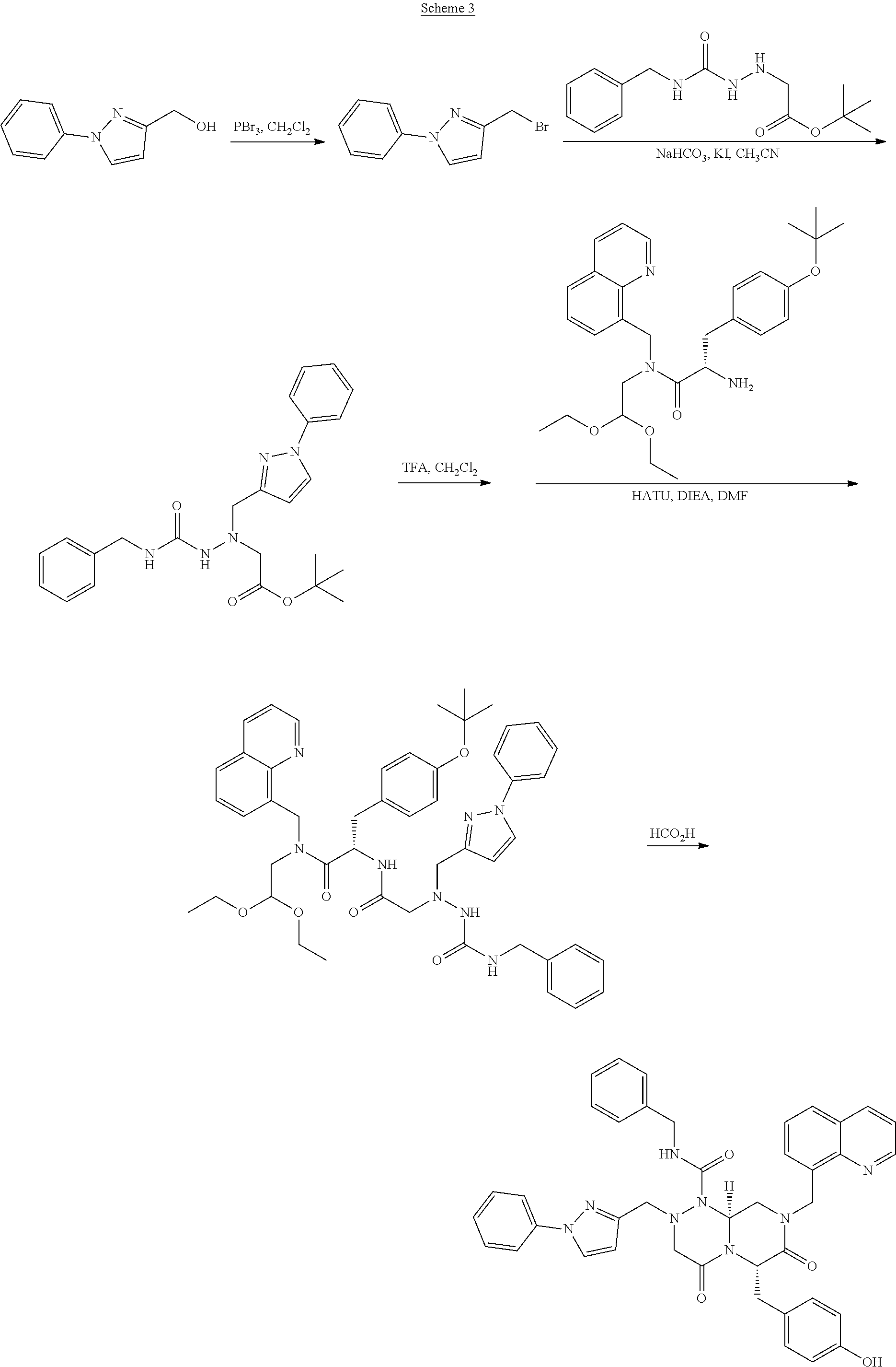

### Step 1: 3-(bromomethyl)-1-phenyl-1H-pyrazole

To a solution of (1-phenyl-1H-pyrazol-3-yl)methanol (0.2 g, 1.15 mmol) in anhydrous CH₂Cl₂ (3 mL) at 0° C. was added PBr₃ (0.11 mL, 1.15 mmol) dropwise. The reaction mixture was stirred at 0° C. under argon for 2 hours, and poured into sat'd NaHCO₃ (15 mL) and CH₂Cl₂ (15 mL). The aqueous layers were extracted with CH₂Cl₂ (15 mL). The organic extracts were dried (Na₂SO₄), evaporated and purified by silica gel chromatography using 15% EtOAc/hexane afforded the title compound (91 mg) as a yellow oil. ¹H-NMR (300 MHz, CDCl₃) δ 7.9 (t, J=3 Hz, 1H), 7.7-7.3 (m, 5H), 6.5 (d, J=3 Hz, 1H), 4.6 (d, J=9 Hz, 2H).

### Step 2-5: (6S,9aS)—N-benzyl-6-(4-hydroxybenzyl)-4,7-dioxo-2-((1-phenyl-1H-pyrazol-3-yl)methyl)-8-(quinolin-8-ylmethyl)octahydro-1H-pyrazino[2,1-c][1,2,4]triazine-1-carboxamide

The title compound 3 as a white solid was obtained, according to the procedures disclosed in US 2021/0317123 A1. MS (ESI): m/z 707.4 (M+H)⁺; analytical HPLC: 15.4 min (99% pure).

### Example 6

### Synthesis of (6S,9aS)—N-benzyl-6-(4-hydroxybenzyl)-4,7-dioxo-2-((1-phenyl-1H-pyrazol-4-yl)methyl)-8-(quinolin-8-ylmethyl)octahydro-1H-pyrazino[2,1-c][1,2,4]triazine-1-carboxamide (4)

Compound 4 as a white solid after lyophilization was obtained, according to Scheme 4, using the procedure described in Example 3. MS (ESI): m/z 707.4 (M+H)⁺; analytical HPLC: 15.2 min (99% pure).

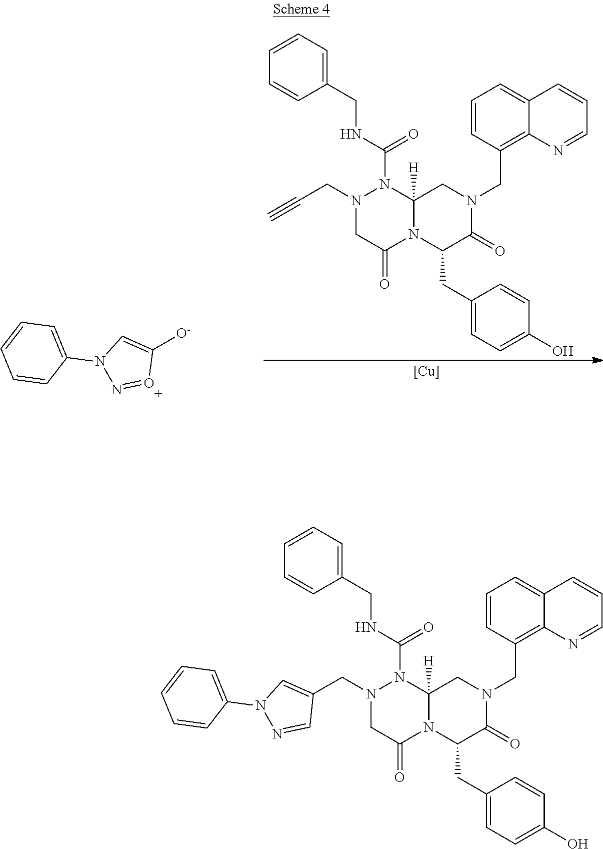

### Example 7

### Biological Characterization of Exemplary Compounds of the Present Invention in the Art-Recognized SuperTOPFLASH Cell-Based Luciferase Assay

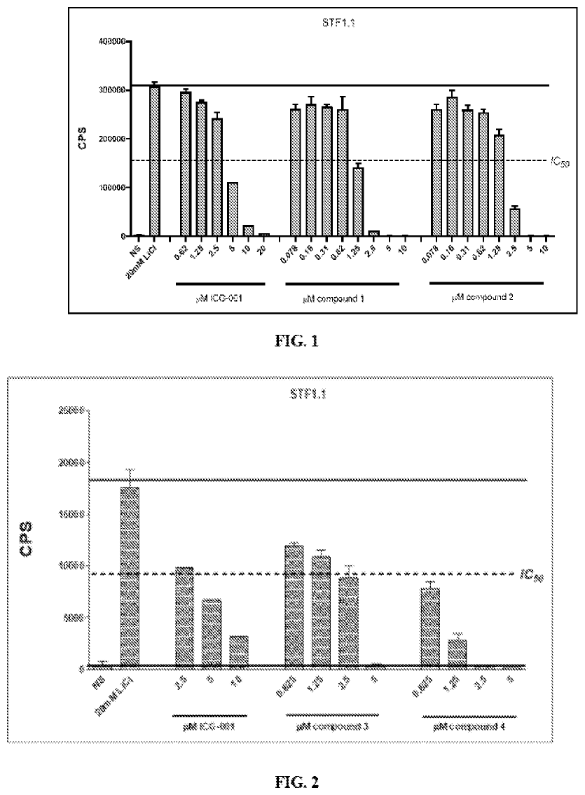

FIG. 1 shows, according to non-limiting aspects of the present invention, results of a SuperTOPFLASH cell-based luciferase assay (Wnt-driven Luciferase Activity in Stably Transfected Cell Line, Hek293, STF1.1), comparing the CBP/β-Catenin inhibition activities of two exemplary 1,4-pyrazole containing compounds, 1 (wherein Z₂ and Z₃ are H) and 2 (wherein Z₂ is Br, and Z₃ is H) of the present invention; each compared in concentrations between 0.078 μM and 10 μM, with the art-recognized specific CBP/β-Catenin inhibitor ICG-001 used as a positive control (at 0.62, 1.25, 2.5, 5, 10 and 20 μM). As is readily apparent, the two inventive pyrazole-containing compounds generally have greater potency than that of ICG-001, and compound 1 has greater potency than that of compound 2 in this assay.

FIG. 2 shows, according to non-limiting aspects of the present invention, results of a SuperTOPFLASH cell-based luciferase assay (Wnt-driven Luciferase Activity in Stably Transfected Cell Line, Hek293, STF1.1), comparing the CBP/β-Catenin inhibition activities of two exemplary pyrazole containing compounds 3 (1,3-pyrazole, wherein Z₂ and Z₃ are H) and 4 (1,4-pyrazole, wherein Z₂ and Z₃ are H) of the present invention; each compared in concentrations between 0.625 μM and 5 μM, with the art-recognized specific CBP/β-Catenin inhibitor ICG-001 used as a positive control (at 2.5, 5 and 10 μM). As is readily apparent, the two inventive pyrazole-containing compounds generally have greater potency than that of ICG-001, and compound 4 has greater potency than that of compound 3 in this assay.

According to the results shown in FIGS. 1 and 2, the representative 1,4-disubstituted pyrazole-containing compounds of the present invention were determined, in general, to have greater potency in this assay.

The invention and the manner and process of making and using it, are now described in such full, clear, concise, and exact terms as to enable any person skilled in the art to which it pertains, to make and use the same.

It will be appreciated that, although specific embodiments of the invention have been described herein for the purposes of illustration, various modifications may be made without departing from the spirit and scope of the invention. Accordingly, the invention is not limited except by the appended claims.

All of the above U.S. patents, U.S. patent application publications, U.S. patent applications, foreign patents, foreign patent applications and non-patent publications referred to in this specification, are incorporated herein by reference.

While certain aspects have been described and shown in the accompanying drawings, it is to be understood that such aspects are merely illustrative of and not restrictive on the broad invention, and that the invention is not limited to the specific constructions and arrangements shown and described, since various other modifications may occur to those of ordinary skill in the art. The description is thus to be regarded as illustrative instead of limiting.

What is claimed is:

1. A compound of formula (Ia): 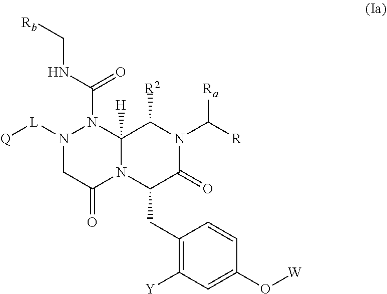
and pharmaceutically acceptable salts thereof, wherein: Rₐ is hydrogen or —CH₃; R_{b} is a monocyclic aryl group having 5 to 7 ring members, which may have 1 to 2 heteroatoms selected from nitrogen, oxygen or sulfur, and which may have one or more substituents selected from a group consisting of halide, cyano, and lower alkyl, and —OC₁-C₆ alkyl; R is a phenyl group; a substituted phenyl group having one or more substituents wherein the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl groups; a benzyl group; a substituted benzyl group with one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; a monocyclic aryl group having 5 to 7 ring members, which may have 1 to 2 heteroatoms selected from nitrogen, oxygen or sulfur, having one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; or a bicyclic aryl group, or substituted bicyclic aryl, having 8 to 11 ring members, which may have 1 to 3 heteroatoms selected from nitrogen, oxygen or sulfur, where the substituted bicyclic aryl may have one or more substituents independently selected from deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; R² is hydrogen, or —CH₃; Y is selected from: hydrogen, deuterium, or halogen; W is hydrogen, phosphate or phosphate salt, an ester of an alkyl acid or of a fatty acid; L is —CH₂—, —CF₂—, or —C(CH₃)₂—; and Q is a 5-membered nitrogen-containing heteroaryl selected from: 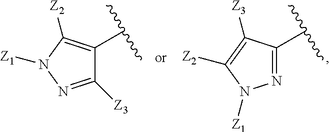
wherein Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C₆ alkyl, —OC₁-C₆ alkyl-C(O)NH—OH, —NH₂, —C(O)NH—C₁-C₆ alkyl-heteroaryl, —NHC(O)C₁-C₆ alkyl-C(O)NH—OH, heteroaryl, cycloalkyl, heterocycloalkyl, Z₂ is independently selected from hydrogen, deuterium, halogen, C₁-C₄ alkyl, aryl, C₁-C₄ alkyl-C(O)—, aryl-C(O)—, C₁-C₄ alkyl-S—, aryl-S—, —C(O)O—C₁-C₄ alkyl, —CF₃, Z₃ is independently selected from hydrogen, deuterium, C₁-C₄ alkyl, —C(O)O—C₁-C₄ alkyl, —C(O)NH—C₁-C₄ alkyl.

2. The compound of claim 1, wherein W is the ester of the alkyl acid or of the fatty acid is selected from: 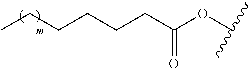
wherein m is 1 to 14,

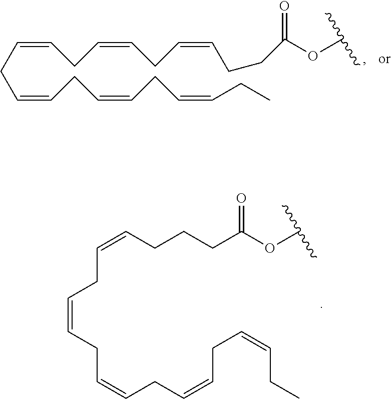 [SMILES: *OC(=O)CC/C=C\C/C=C\C/C=C\C/C=C\C/C=C\C/C=C\CC ; *OC(=O)CCC/C=C\C/C=C\C/C=C\C/C=C\C/C=C\CC]

3. The compound of claim 1, the compound is of the formula (Ib): 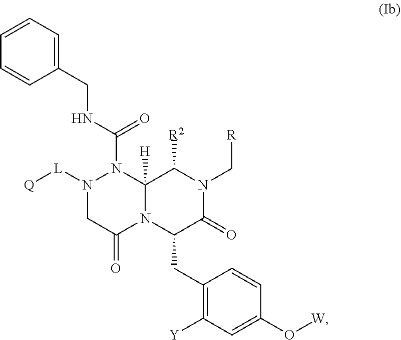
wherein R is as defined above.

4. The compound of claim 3, wherein: L is —CH₂—; Q is 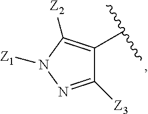
wherein Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C₆ alkyl, —OC₁-C₆ alkyl-C(O)NH—OH, —NH₂, —C(O)NH—C₁-C₆ alkyl-heteroaryl, —NHC(O)C₁-C₆ alkyl-C(O)NH—OH, heteroaryl, cycloalkyl, heterocycloalkyl, Z₂ and Z₃ are independently selected from hydrogen, deuterium.

5. The compound of claim 4, wherein the compound is: 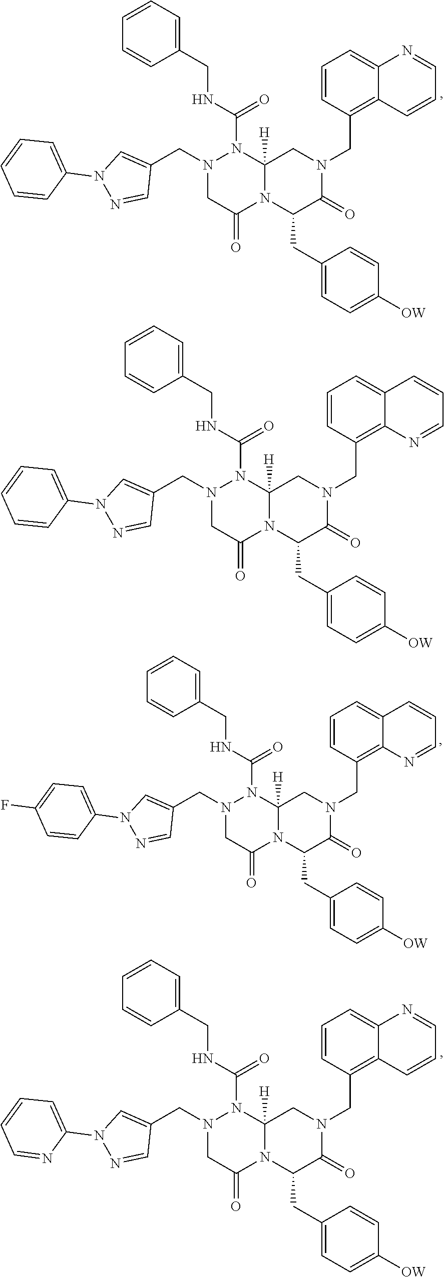 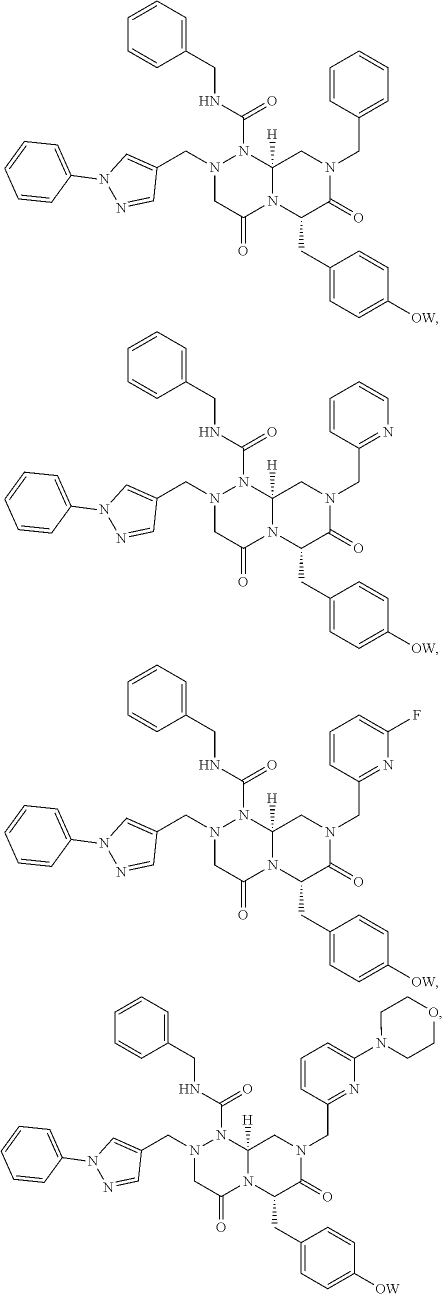 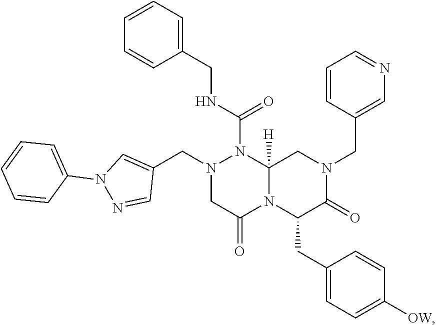
wherein W is as defined above.

6. A composition or pharmaceutical composition comprising the compound of claim 1, and a pharmaceutically acceptable carrier.

7. A method of treating a disease or disorder, comprising administering to a patient or a warm-blooded mammal, having a disease or disorder mediated by CREB binding protein (CBP)/β-catenin signaling, an amount of a compound sufficient to inhibit CBP/catenin signaling, and/or enhance p300/catenin mediated signaling, and wherein the compound is of formula (Ia): 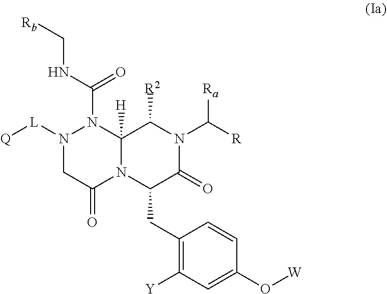
and pharmaceutically acceptable salts thereof, wherein: Rₐ is hydrogen or —CH₃; R_{b} is a monocyclic aryl group having 5 to 7 ring members, which may have 1 to 2 heteroatoms selected from nitrogen, oxygen or sulfur, and which may have one or more substituents selected from a group consisting of halide, cyano, and lower alkyl, and —OC₁-C₆ alkyl; R is a phenyl group; a substituted phenyl group having one or more substituents wherein the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl groups; a benzyl group; a substituted benzyl group with one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; a monocyclic aryl group having 5 to 7 ring members, which may have 1 to 2 heteroatoms selected from nitrogen, oxygen or sulfur, having one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; or a bicyclic aryl group, or substituted bicyclic aryl, having 8 to 11 ring members, which may have 1 to 3 heteroatoms selected from nitrogen, oxygen or sulfur, where the substituted bicyclic aryl may have one or more substituents independently selected from deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; R² is hydrogen, or —CH₃; Y is selected from: hydrogen, deuterium, or halogen; W is hydrogen, phosphate or phosphate salt, an ester of an alkyl acid or of a fatty acid; L is —CH₂—, —CF₂—, or —C(CH₃)—; and Q is a 5-membered nitrogen-containing heteroaryl selected from: 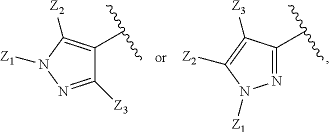
wherein Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C₆ alkyl, —OC₁-C₆ alkyl-C(O)NH—OH, —NH₂, —C(O)NH—C₁-C₆ alkyl-heteroaryl, —NHC(O)C₁-C₆ alkyl-C(O)NH—OH, heteroaryl, cycloalkyl, heterocycloalkyl, Z₂ is independently selected from hydrogen, deuterium, halogen, C₁-C₄ alkyl, aryl, C₁-C₄ alkyl-C(O)—, aryl-C(O)—,
C₁-C₄ alkyl-S—, aryl-S—, —C(O)O—C₁-C₄ alkyl, —CF₃, Z₃ is independently selected from hydrogen, deuterium, C₁-C₄ alkyl, —C(O)O—C₁-C₄ alkyl, —C(O)NH—C₁-C₄ alkyl.

8. The method of claim 7, wherein the amount of the administered compound comprises a therapeutically effective amount.

9. The method of claim 8, wherein the disease or disorder comprises one or more of fibrosis, cancer, neurological conditions, metabolic disorders, skin conditions and aging.

10. The method of claim 9, wherein the metabolic disorder comprises one or more of diabetes and/or fatty liver disease.

11. The method of claim 10, wherein the fatty liver disease comprises one or more of alcoholic hepatic steatosis (ALD), non-alcoholic hepatic steatosis (NAFLD), and/or non-alcoholic steatohepatitis (NASH).

12. The method of claim 9, wherein the fibrosis is fibrosis of the lung, liver, kidney, heart, endometrium, skin or systemic fibrosis.

13. The method of claim 12, wherein the fibrosis comprises fibrosis in a SARS-CoV-2 (COVID-19) patient tissue.

14. The method of claim 9, wherein treating cancer comprises administering the CBP/β-catenin antagonist in combination with, or as an adjunctive therapy with, one or more of cytotoxic and/or directed chemotherapy, and/or radiotherapy, and/or immunotherapy, including checkpoint inhibition, chimeric antigen receptor (CAR-T) and/or CAR-NK.

15. The method of claim 9, wherein the neurological condition comprises one or more of Huntington's (HD), Parkinson's (PD), Alzheimer's (AD), Multiple sclerosis (MS), and/or amyotrophic lateral sclerosis (ALS), muscular dystrophy (MD), and/or spinal muscular atrophy (SMA).

16. The method of claim 9, wherein the skin condition comprises one or more of atopic dermatitis, psoriasis, acne, fibrosis, wounding, scarring, burns, sun or U.V. damage, diabetic ulceration, chronic ulceration, and/or alopecia.

17. The method of claim 16, wherein W is an ester of an alkyl acid or of a fatty acid, and wherein administration comprises topical or transdermal administration.

18. A cosmetic method for treating a skin condition, comprising topically administering to a patient or a warm-blooded mammal, having a skin condition, a cosmeceutically effective amount of a compound of formula (Ia): 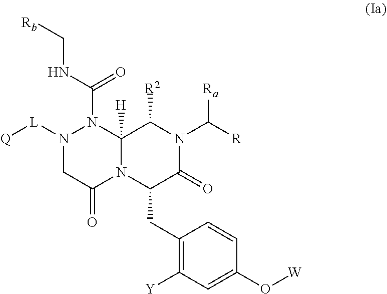
and pharmaceutically acceptable salts thereof, wherein: Rₐ is hydrogen or —CH₃; R_{b} is a monocyclic aryl group having 5 to 7 ring members, which may have 1 to 2 heteroatoms selected from nitrogen, oxygen or sulfur, and which may have one or more substituents selected from a group consisting of halide, cyano, and lower alkyl, and —OC₁-C₆ alkyl; R is a phenyl group; a substituted phenyl group having one or more substituents wherein the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl groups; a benzyl group; a substituted benzyl group with one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; a monocyclic aryl group having 5 to 7 ring members, which may have 1 to 2 heteroatoms selected from nitrogen, oxygen or sulfur, having one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; or a bicyclic aryl group, or substituted bicyclic aryl, having 8 to 11 ring members, which may have 1 to 3 heteroatoms selected from nitrogen, oxygen or sulfur, where the substituted bicyclic aryl may have one or more substituents independently selected from deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; R² is hydrogen, or —CH₃; Y is selected from: hydrogen, deuterium, or halogen; W is hydrogen, phosphate or phosphate salt, an ester of an alkyl acid or of a fatty acid; L is —CH₂—, —CF₂—, or —C(CH₃)₂—; and Q is a 5-membered nitrogen-containing heteroaryl selected from: 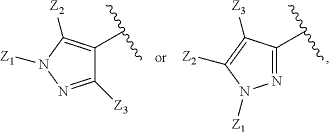
wherein Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C₆ alkyl, —OC₁-C₆ alkyl-C(O)NH—OH, —NH₂, —C(O)NH—C₁-C₆ alkyl-heteroaryl, —NHC(O)C₁-C₆ alkyl-C(O)NH—OH, heteroaryl, cycloalkyl, heterocycloalkyl, Z₂ is independently selected from hydrogen, deuterium, halogen, C₁-C₄ alkyl, aryl, C₁-C₄ alkyl-C(O)—, aryl-C(O)—, C₁-C₄ alkyl-S—, aryl-S—, —C(O)O—C₁-C₄ alkyl, —CF₃, Z₃ is independently selected from hydrogen, deuterium, C₁-C₄ alkyl, —C(O)O—C₁-C₄ alkyl, —C(O)NH—C₁-C₄ alkyl,
wherein W is an ester of an alkyl acid or of a fatty acid, preferably, wherein the ester of the alkyl acid or of the fatty acid is selected from: 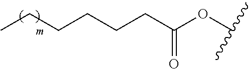
wherein m is 1 to 14,

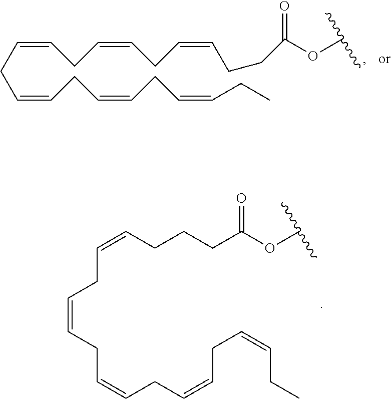 [SMILES: *OC(=O)CC/C=C\C/C=C\C/C=C\C/C=C\C/C=C\C/C=C\CC ; *OC(=O)CCC/C=C\C/C=C\C/C=C\C/C=C\C/C=C\CC]

19. The method of claim 18, wherein the skin condition comprises one or more aging skin conditions selected from wrinkles, hyperpigmentation, redness, rosacea, dryness, cracking, loss of vibrance, loss of elasticity, thinning, loss of vibrance, scarring, acne, sun damage, hair loss, loss of hair coloration, reduced cuticle growth, reduced nail growth.

20. A method for efficiently synthesizing a clinical grade drug, comprising use, in a penultimate, or last reaction step under GMP conditions, of an intermediate 2-propynyl-compound to form a clinical grade pyrozole derivative via 3+2 cycloaddition.

21. A method for enhancing vaccine efficacy, comprising, administering to a subject, prior to, and/or during, and/or after vaccination, an amount of a compound sufficient to inhibit CBP/E-catenin mediated signaling and/or enhance p300/catenin mediated signaling, wherein the compound is of formula (Ia): 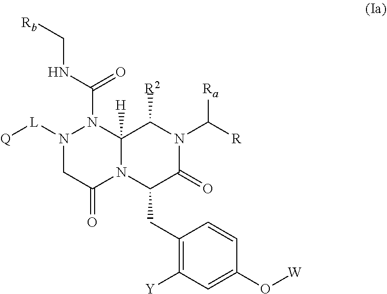
and pharmaceutically acceptable salts thereof, wherein: Rₐ is hydrogen or —CH₃; R_{b} is a monocyclic aryl group having 5 to 7 ring members, which may have 1 to 2 heteroatoms selected from nitrogen, oxygen or sulfur, and which may have one or more substituents selected from a group consisting of halide, cyano, and lower alkyl, and —OC₁-C₆ alkyl; R is a phenyl group; a substituted phenyl group having one or more substituents wherein the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl groups; a benzyl group; a substituted benzyl group with one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; a monocyclic aryl group having 5 to 7 ring members, which may have 1 to 2 heteroatoms selected from nitrogen, oxygen or sulfur, having one or more substituents where the one or more substituents are independently selected from one or more of deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; or a bicyclic aryl group, or substituted bicyclic aryl, having 8 to 11 ring members, which may have 1 to 3 heteroatoms selected from nitrogen, oxygen or sulfur, where the substituted bicyclic aryl may have one or more substituents independently selected from deuterium, amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; R² is hydrogen, or —CH₃; Y is selected from: hydrogen, deuterium, or halogen; W is hydrogen, phosphate or phosphate salt, an ester of an alkyl acid or of a fatty acid; L is —CH₂—, —CF₂—, or —C(CH₃)₂—; and Q is a 5-membered nitrogen-containing heteroaryl selected from: 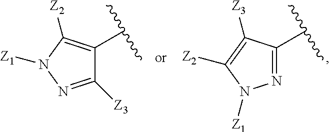
wherein Z₁ is selected from aryl, heteroaryl, each of which is substituted by 0-4 substituents independently selected from hydrogen, deuterium, halogen, C₁-C₄ alkyl, C₁-C₃ haloalkyl, —OH, —OC₁-C₆ alkyl, —OC₁-C₆ alkyl-C(O)NH—OH, —NH₂, —C(O)NH—C₁-C₆ alkyl-heteroaryl, —NHC(O)C₁-C₆ alkyl-C(O)NH—OH, heteroaryl, cycloalkyl, heterocycloalkyl, Z₂ is independently selected from hydrogen, deuterium, halogen, C₁-C₄ alkyl, aryl, C₁-C₄ alkyl-C(O)—, aryl-C(O)—, C₁-C₄ alkyl-S—, aryl-S—, —C(O)O—C₁-C₄ alkyl, —CF₃, Z₃ is independently selected from hydrogen, deuterium, C₁-C₄ alkyl, —C(O)O—C₁-C₄ alkyl, —C(O)NH—C₁-C₄ alkyl.

22. The method of claim 21, wherein the amount of the administered compound comprises a therapeutically effective amount.

23. The method of claim 21, wherein enhancing vaccine efficacy comprises one or more of: increased levels of vaccine antigen-specific antibodies; an increase in the percent protection afforded; an increase in the number or and/or persistence of differentiated memory T-cells; and/or an increase in the duration of protection.

24. The method of claim 21, wherein inhibiting the CBP/E-catenin mediated signaling and/or enhancing the p300/catenin mediated signaling comprises one or more of: metabolic maintenance of cell asymmetry following division in activated T cells of the subject; enhancing antigen-specific immunity by increasing the number and/or persistence of differentiated memory T-cells; and/or enhancing the presentation of antigens to T-cells by antigen presenting cells to enhance cooperativity between the innate and acquired immune systems.

25. The method of claim 21, wherein the vaccination, comprises administration of an anti-viral vaccine.

26. The method of claim 21, wherein the vaccination comprises administration of an anti-viral vaccine selected from influenza, SARS, SARS-CoV-2, HPV-A, HPV-B, and/or Herpes Zoster.

27. The method of claim 21, wherein the subject is a human having an age of 55-75 yr, 55-85 yr, ≥50 yr, ≥60 yr, or ≥65 yrs).

28. The method of claim 21, wherein administration comprises: administration as a primer before vaccination; and/or co-administration with vaccination; and/or administration or co-administration subsequent to vaccine.
